(19) United States
(12) Patent Application Publication
(10) Pub. No.: US 2004/0083062 A1
(43) Pub. Date: Apr. 29, 2004

(54) Method for the processing of several different data structures

(76) Inventor: Maria Athelogou, Munchen (DE)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/332,521

(22) PCT Filed: Jul. 6, 2001
(86) PCT No.: PCT/EP01/07795

(30) Foreign Application Priority Data
Jul. 7, 2000 (DE) 100330215

(51) Int. Cl. G06F 17/20; G06F 19/00; G01N 33/48; G01N 33/50

(52) U.S. Cl. 702/20; 704/1

(57) ABSTRACT

What is disclosed is a method for processing data structures of different structures and contents by means of networked semantic units, which method includes the steps of: the steps of: acquisition of data wherefrom the different data structures are derivable, wherein respective data structures are represented in respective networked networks of semantic structure units; and generating, analyzing, modifying, deleting and/or storing the semantic structure units and/or networking them based on the acquired data by using a knowledge base comprised of a network of semantic knowledge units. Herein in iterative steps semantic structure units and/or the networking thereof are classified and a specific processing may be activated thanks to this classification, which specific processing modifies a respective semantic structure unit and/or a particular partial network.

The present invention relates to a computer-implemented method for object-oriented acquisition and processing of several data structures of identical or different contents and in particular of genetic, biological, bio-medical and biochemical data structures, and in particular relates to a computer-implemented method which may be used for pattern recognition, grouping of single data, i.e. object extraction, and simulation with the aid of the data structures to be processed.

In genetics, the expression “sequence analysis” is used to designate the positionally correct identification of monomer units in one macromolecular chain of a nucleic acid, of a protein, etc. The expression “sequence analysis” thus relates to a chemical analytical method.

The expression “comparative sequence analysis”, on the other hand, relates to the precondition that the sequence of structural units as such is already known for the set of macromolecules in question, and that the informational relations between different sequences are to be subjected to a comparative analysis.

Sequence analysis methods in the prior art are in most cases restricted, for example, to reading base triplets in one long row of consecutive bases, without this process having an underlying knowledge intelligence for the acquisition and allocation of respective structures. This results in a high error rate in the acquisition of a respective genetic information. Furthermore it is difficult in a comparative analysis and in particular in a comparative sequence analysis to achieve an allocation of a recognized structure to a genetic information. This is in particular due to the natural diversity of genetic information.

In addition, up to now only conditionally dynamic databases have been used, which allow for processing relating to a set problem within the corresponding database. As a result, dynamic, automatic networking of all those databases containing genetic information is only conditionally possible. Just the same in the case of networking, smooth flow of information both between different databases and also locally within respective ones of the databases in the sense of simultaneous and autonomous is only conditionally possible.

The present invention was conceived with a view to the above described problems.

Accordingly it is the object of the present invention to furnish a method for processing several different data structures and the networking thereof, which is capable of ensuring smooth flow of information both between different databases and also locally within respective ones of the databases in all directions.

This object is achieved by the measures specified in claim 1.

To be more precise, in accordance with the invention a method is created for processing data structures of different structures and contents by means of networked semantic units, which method includes the steps of: acquisition of data wherefrom the different data structures are derivable, wherein respective data structures are represented in respective networked networks of semantic structure units; and generating, analyzing, modifying, deleting and/or storing the semantic structure units and/or networking them based on the acquired data by using a knowledge base comprised of a network of semantic knowledge units, wherein in iterative steps semantic structure units and/or the networking thereof are classified and a specific processing may be activated thanks to this classification, which specific processing modifies a respective semantic structure unit and/or a particular partial network.

Thanks to the circumstance that respective different data structures are represented in respective different networks of structure units, there accordingly is a possibility of classifying and processing both each data structure by itself and also the data structures among each other by using the network of semantic knowledge units. These classifications and processings of the data structures both by themselves and among each other may be carried out simultaneously. In this way it is possible to take into account dependencies or relationships both within and between the respective networks during processing. This creates the possibility of ensuring information flows in all directions both within a respective network of structure units and between all respective networks of semantic structure units which furthermore may be placed in mutual interaction.

In accordance with a development of the present invention, each of the semantic networks of structure units includes a hierarchy with hierarchy levels of superordinate, subordinate and neighboring semantic structure units.

In accordance with another development of the present invention, superordinate semantic structure units comprise subordinate structure units from different networks of semantic structure units.

In accordance with another development of the present invention, data is acquired in one or several arbitrary hierarchy levels.

In accordance with another development of the present invention, the networks of semantic structure units overlap each other.

In accordance with another development of the present invention, in step [b] a networking between networks of semantic structure units is changed.

In accordance with another development of the present invention, in step [b] a networking between the network of semantic knowledge units and the networks of semantic structure units is changed.

In accordance with another development of the present invention, the several different data structures are interrelated biological, biochemical, bio-medical and/or genetic data structures.

In accordance with another development of the present invention, the several different data structures are DNA data structures, RNA data structures and/or protein data structures that are each represented in a network of semantic structure units.

In accordance with another development of the present invention, a classification is carried out by comparing, allocating, feeding back and deriving the several different data structures in such a manner that respective networks of semantic structure units of the several different data structures are classified by means of the network of semantic knowledge units themselves and in dependence on this classification relationships and networkings within the respective networks of semantic structure units of the several different data structures are generated, and the respective networks of semantic structure units of the several different data structures are classified with respect to each other by means of the network of semantic knowledge units, and relationships and networkings between the respective networks of semantic structure units of the several different data structures are generated in dependence on this classification.

In accordance with another development of the present invention, networking between the respective ones of the networks of semantic structure units of the several different data structures takes place with the aid of a classification of information flows from a network of semantic structure units of the DNA data structures to a network of semantic structure units of the RNA data structures to a network of semantic structure units of the protein data structures and similar regressive processes.

In accordance with another development of the present invention, semantic structure units are structure objects, linking objects linking semantic structure units, or networks/partial networks of semantic structure units.

In accordance with another development of the present invention, the semantic structure objects are present in a super-structure object hierarchy, sub-structure object hierarchy and neighbor-structure object hierarchy in the networks of semantic structure units, with respective linking objects defining respective relationships between respective structure objects.

In accordance with another development of the present invention, the relationships include neighborhood relationships, similarity relationships, relationships with super-structure objects, and relationships with sub-structure objects.

In accordance with another development of the present invention, DNA sequences in a network of semantic structure units of DNA data structures are classified in that nucleotide structure units are grouped in sequences of three into newly formed codogen structure units and relationships between semantic structure units are formed, wherein the relationships include neighborhood relationships, similarity relationships, relationships with super-structure objects and relationships with sub-structure objects.

In accordance with another development of the present invention, RNA sequences are classified in a network of semantic structure units of RNA data structures in that nucleotide structure units are grouped in sequences of three into newly formed codon structure units and relationships between semantic structure units are formed, wherein the relationships include neighborhood relationships, similarity relationships, relationships with super-structure objects and relationships with sub-structure objects.

In accordance with another development of the present invention, relationships between DNA sequences in a network of semantic structure units of DNA data structures and RNA sequences in a network of semantic structure units of RNA data structures are formed by semantic structure units describing an allocation of semantic DNA structure units to semantic RNA structure units.

In accordance with another development of the present invention, a classification of semantic structure units describing amino acids is carried out in a network of semantic structure units of protein data structures in such a manner that the semantic structure units describing amino acids are grouped, semantic structure units for the formed amino acid groups are formed, and relationships between the semantic structure units are formed, wherein the relationships include neighborhood relationships between the semantic structure units describing amino acids and similarity relationships between the semantic structure units describing amino acids, and wherein semantic structure units are generated and defined which describe relationships with semantic enzyme structure units, and semantic structure units are generated which describe relationships with super-structure objects.

In accordance with another development of the present invention, the super-structure objects are semantic protein structure units.

In accordance with another development of the present invention, local processes are classified as semantic units describing processes of a transformation of biological information.

In accordance with another development of the present invention, the local processes are described by copying a DNA structure unit to an RNA structure unit; fusing semantic structure units for nucleotide triplets into semantic structure units for an operon; separating semantic units for nucleotide triplet groups into single semantic structure units for a nucleotide triplet; regrouping semantic structure units for nucleotide triplets of a semantic structure unit for an operon into another semantic structure unit for an operon; deleting semantic structure units for nucleotide triplets within a semantic structure unit for an operon; and allocating semantic structure units for nucleotide triplets to semantic structure units for an operon.

In accordance with another development of the present invention, local processes are classified as semantic units describing processes of a transformation of biochemical and genetic information.

In accordance with another development of the present invention, the local processes are described by allocating semantic structure units for amino acids to a semantic structure unit for an m-RNA; fusing semantic structure units for amino acids into higher semantic structure units within a protein and generating its allocation to semantic structure units for metabolic processes; separating semantic structure units for amino acid groups into single semantic structure units for an amino acid; regrouping semantic structure units for amino acids or amino acid groups; deleting semantic structure units for amino acids within a partial network describing a semantic structure unit for a protein; allocating semantic structure units for amino acids to the semantic structure units for proteins; and forming semantic linking objects between the semantic structure units for amino acids, the semantic structure units for amino acid groups, and the semantic structure units for proteins.

In accordance with another development of the present invention, semantic linking objects are formed which describe an allocation of a semantic structure unit for a t-RNA to different amino acids in protein formation.

In accordance with another development of the present invention, semantic structure units are formed which describe local processes of copying, of searching, of prompting, of selecting, and of docking as semantic processing objects.

In accordance with another development of the present invention, the following processes may be performed within a data structure: fusing two or more codon structure units into a new super-structure unit, wherein at the same time the nucleotide structure units pertaining thereto are divided and new neighborhood relationships between these are generated; fusing two or more codon or operon structure units into a larger structure unit by generating a new hierarchy level, wherein the corresponding codon or operon structure units form the sub-structure units of this new, larger structure unit; incorporating two or more new structure units within a network of semantic structure units in such a way that upon generation of a new incorporation, a structure unit becomes a sub-structure unit of another structure unit; separating a structure unit from its super-structure unit by generating a new kind of a neighborhood relationship with the former super-structure unit; newly allocating a structure unit to a new sub-structure unit; and regrouping a structure unit from one group of structure units to another group of structure units.

In accordance with another development of the present invention, a classification algorithm is started by string-matching, wherein knowledge units are characterized by particular strings and the classification algorithm recognizes fragments in the several different data structures having identical or similar strings, and generates classification connections with corresponding knowledge units of semantic structure units which correspond to these fragments, whereby in turn new algorithms may be called up.

In accordance with another development of the present invention, classification is carried out with the aid of attributes.

In accordance with another development of the present invention, the attributes include the strings code, content, length, variance, texture and position.

In accordance with another development of the present invention, classification is carried out with the aid of neighborhood relationships.

In accordance with another development of the present invention, the neighborhood relationships include what neighbors, what relationship with the neighbors, what super-units, what sub-units and what relationships with the super-units and/or sub-units exist.

In accordance with another development of the present invention, semantic structure units are generated which are merely allocated a fictitious meaning in a classification by means of the network of semantic knowledge units.

In accordance with another development of the present invention, the semantic structure units having merely been allocated a fictitious meaning are taken into consideration in another classification.

Additional advantageous developments of the present invention are subject matters of the appended claims.

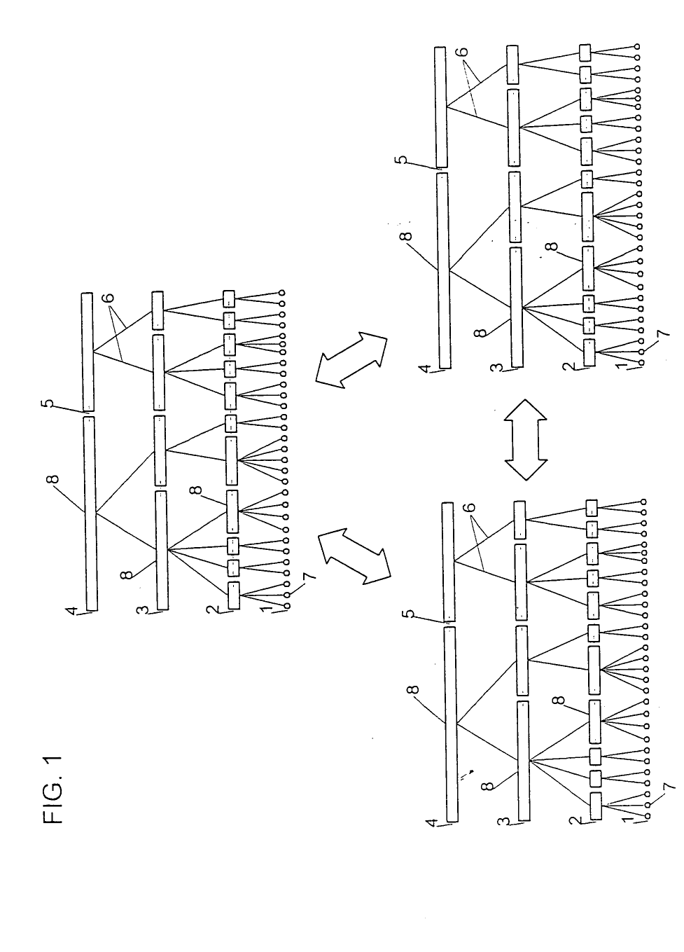

FIG. 1 is a schematic representation of several hierarchical networks of structure units in accordance with an embodiment of the present invention;

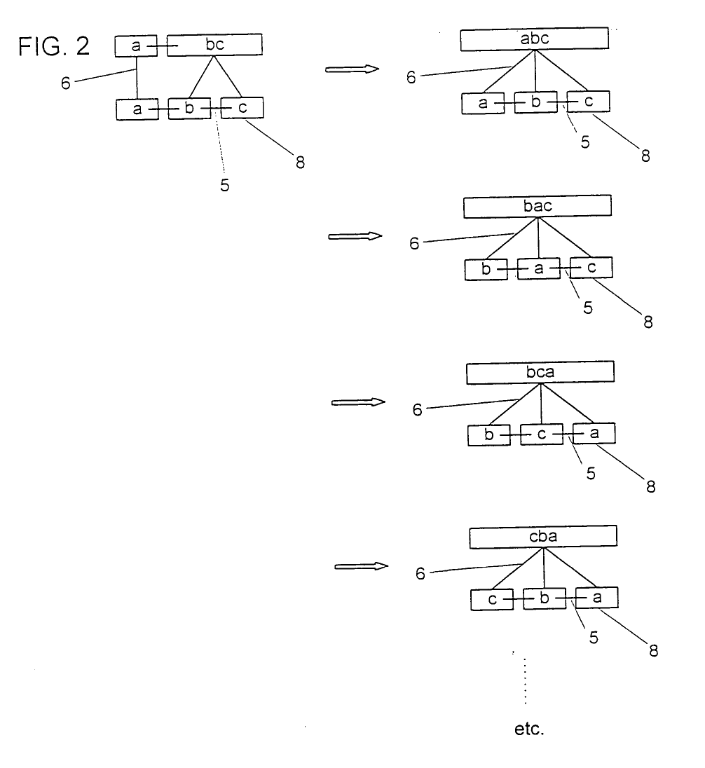

FIG. 2 shows a local operation “fusion” within the hierarchical networks of structure units with the aid of a respective network section in accordance with the practical example of the present invention;

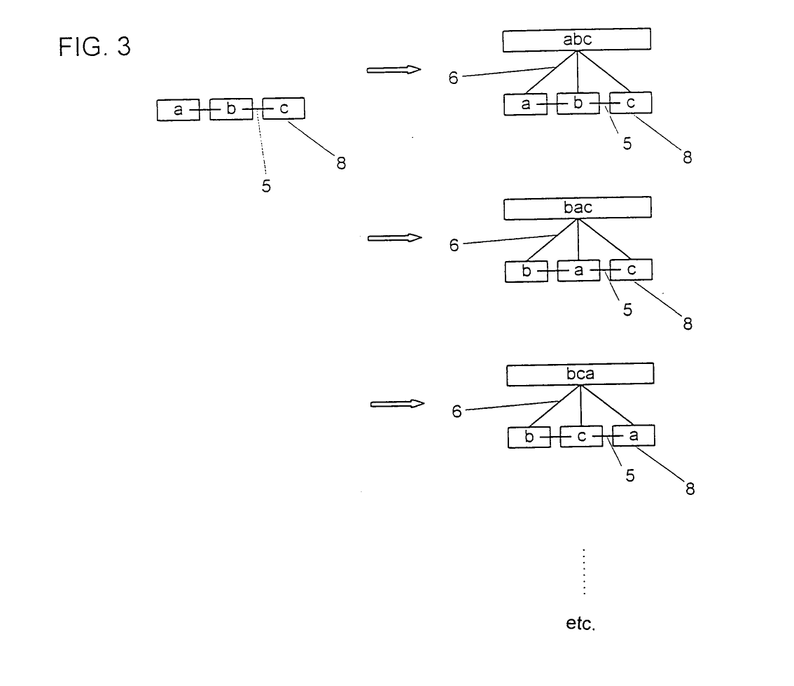

FIG. 3 shows a local operation “founding” within the hierarchical networks of structure units with the aid of a respective network section in accordance with the practical example of the present invention;

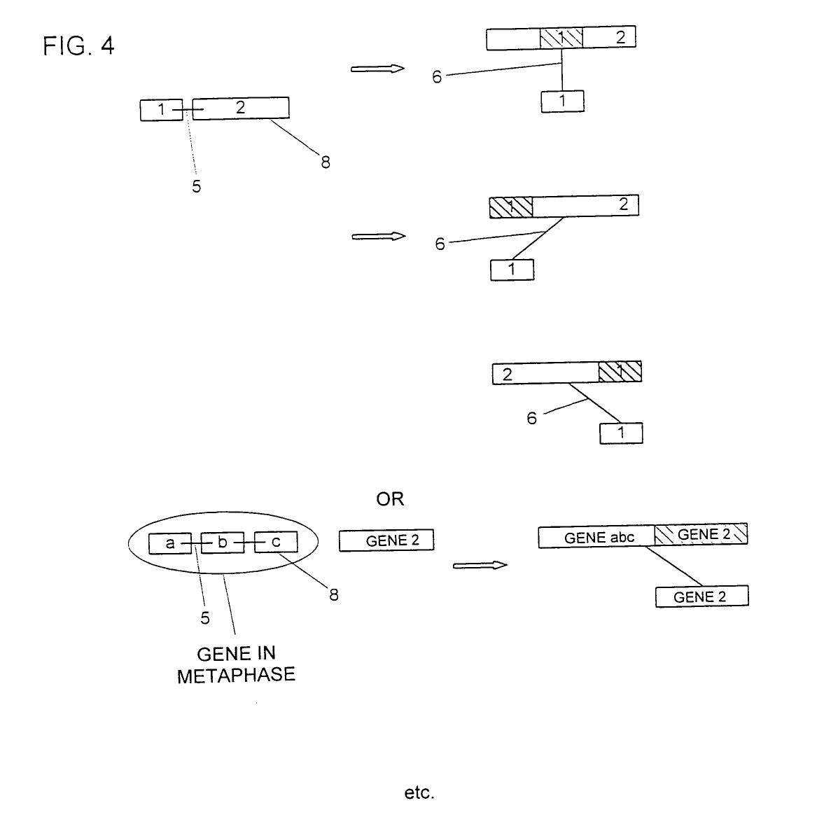

FIG. 4 shows a local operation “insertion of a neighbor as a sub-structure object” within the hierarchical networks of structure units with the aid of a respective network section in accordance with the practical example of the present invention;

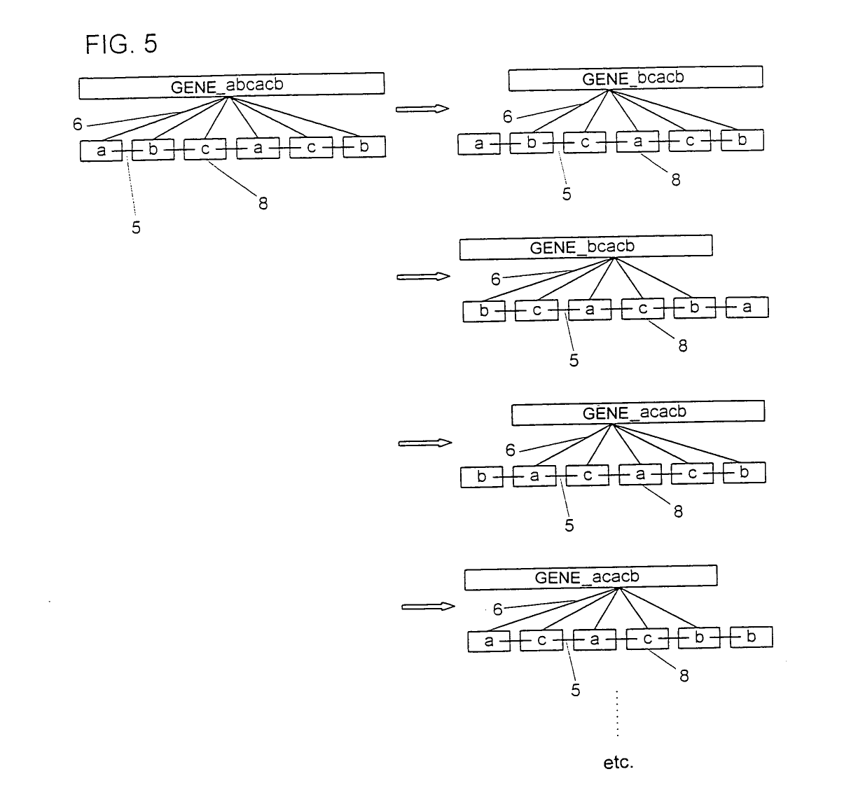

FIG. 5 shows a local operation “exclusion” within the hierarchical networks of structure units with the aid of a respective network section in accordance with the practical example of the present invention;

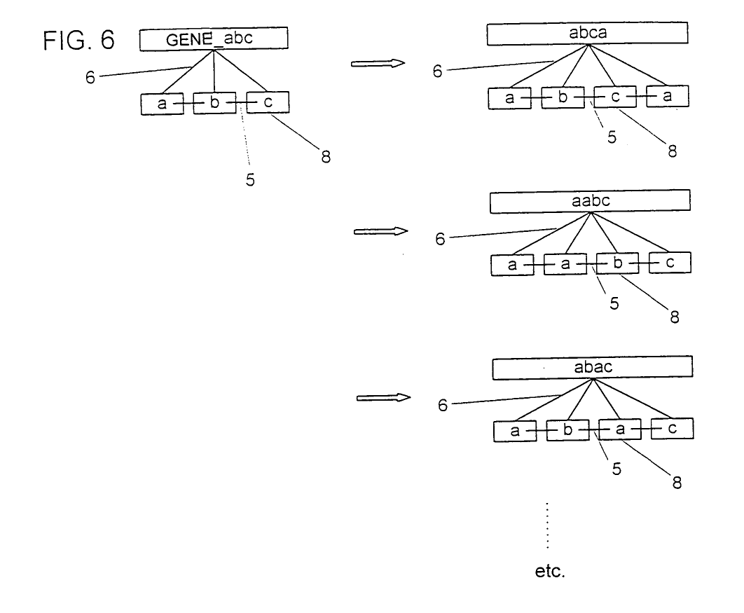

FIG. 6 shows a local operation “insertion of a new sub-structure object” within the hierarchical networks of structure units with the aid of a respective network section in accordance with the practical example of the present invention;

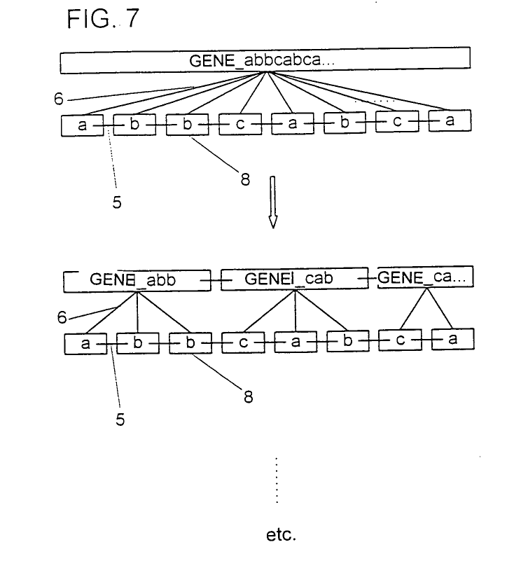

FIG. 7 shows a local operation “division” within the hierarchical networks of structure units with the aid of a respective network section in accordance with the practical example of the present invention;

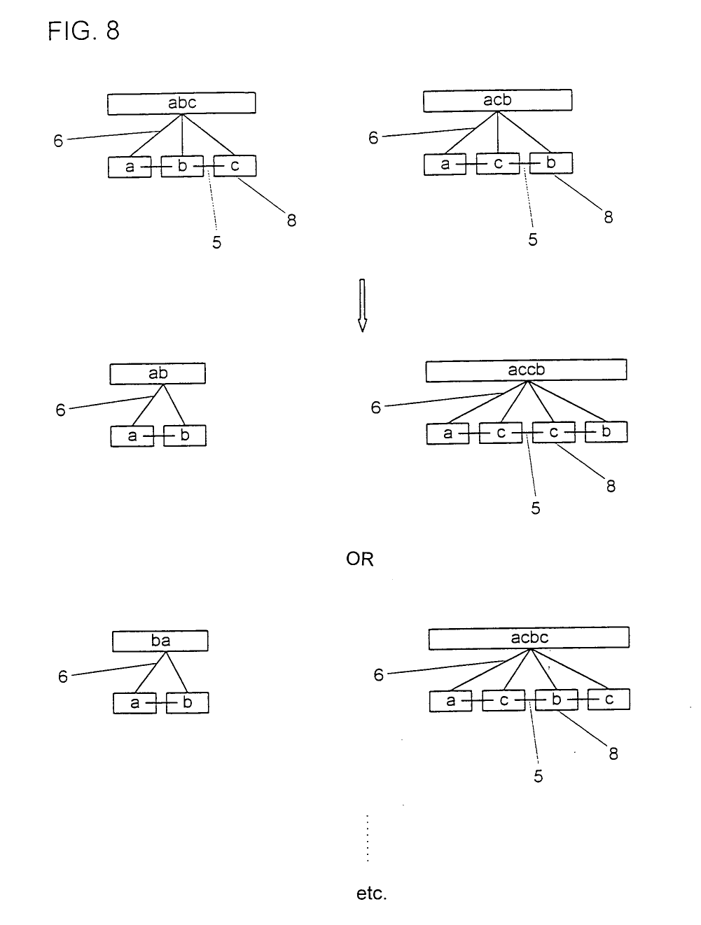

FIG. 8 shows a local operation “regrouping” within the hierarchical networks of structure units with the aid of a respective network section in accordance with the practical example of the present invention;

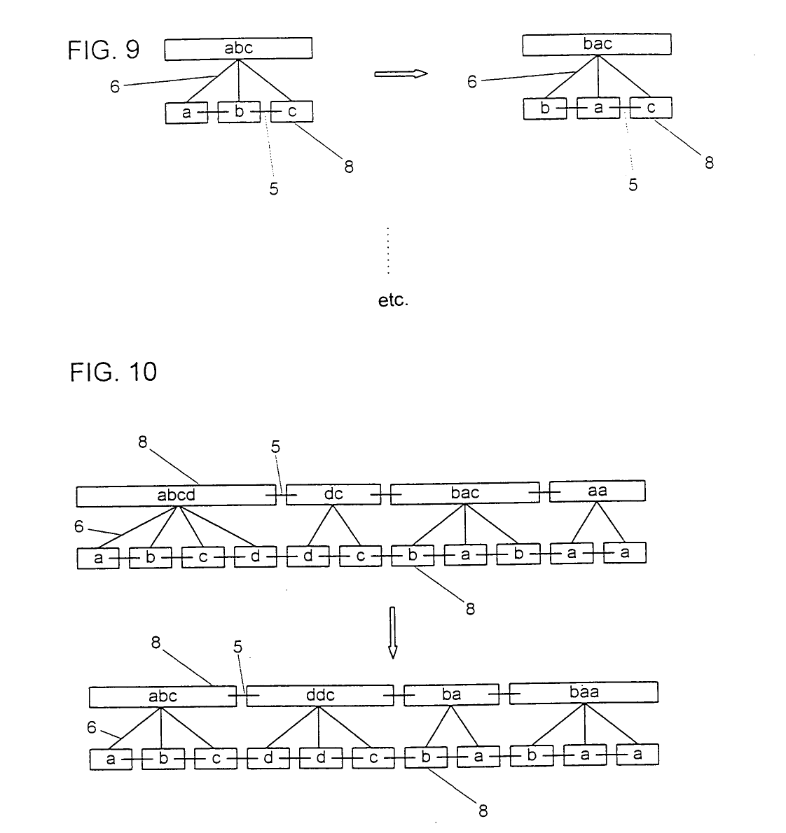

FIG. 9 shows a local operation “jumping/exchanging” within the hierarchical networks of structure units with the aid of a respective network section in accordance with the practical example of the present invention;

FIG. 10 shows a local operation “boundary optimization” within the hierarchical networks of structure units with the aid of a respective network section in accordance with the practical example of the present invention;

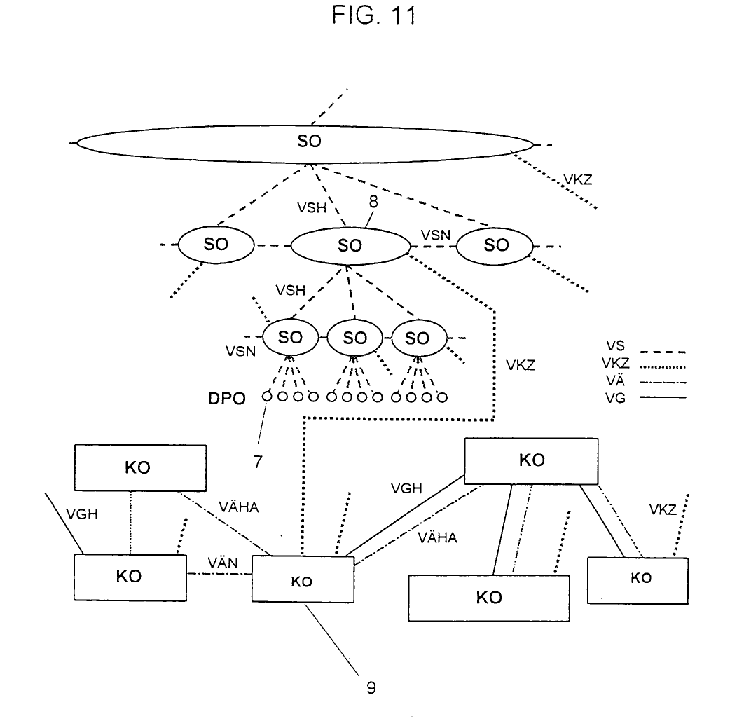

FIG. 11 shows an example of networked environs of a classified structure object and of a corresponding class object in schematic representation;

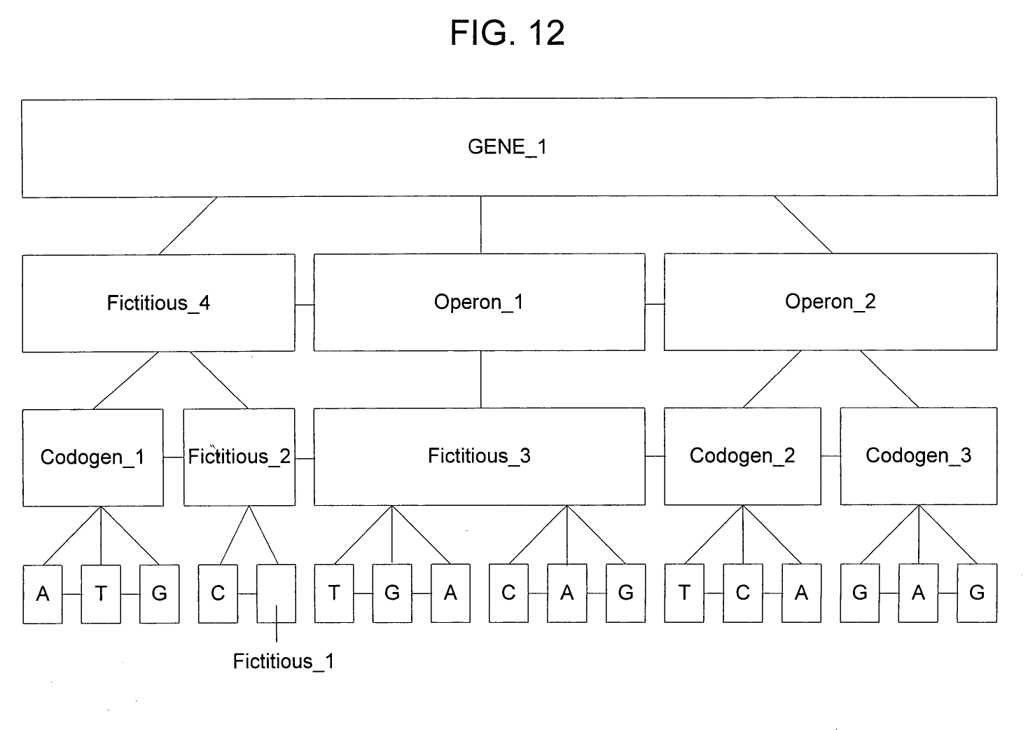

FIG. 12 shows an example of a classified hierarchical network of structure units of a DNA data structure in accordance with the practical example of the present invention;

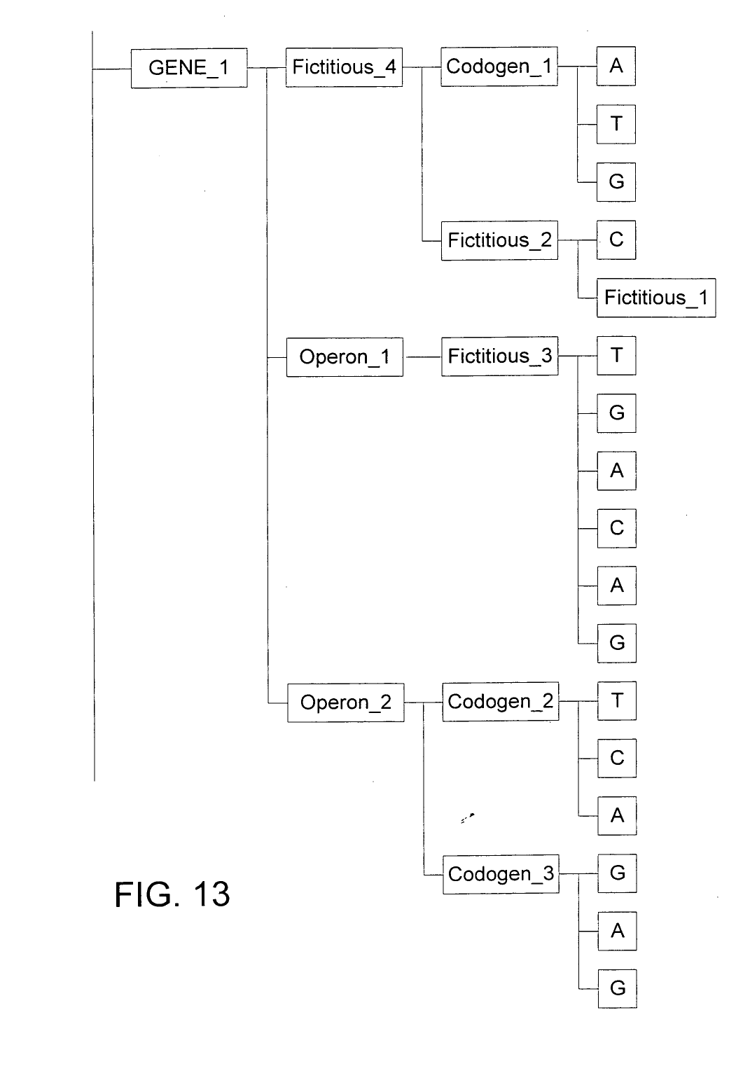

FIG. 13 is a representation of class objects in one grouping hierarchy in accordance with the DNA data structure in FIG. 12;

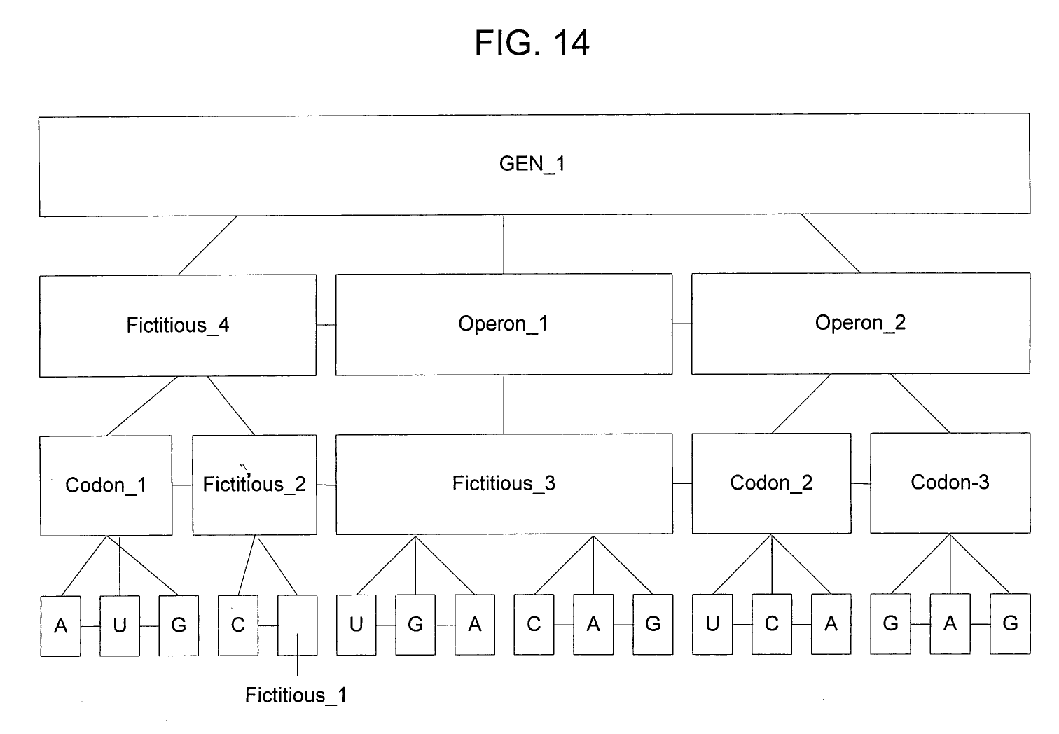

FIG. 14 shows an example of a classified hierarchical network of structure units of an RNA data structure in accordance with the practical example of the present invention;

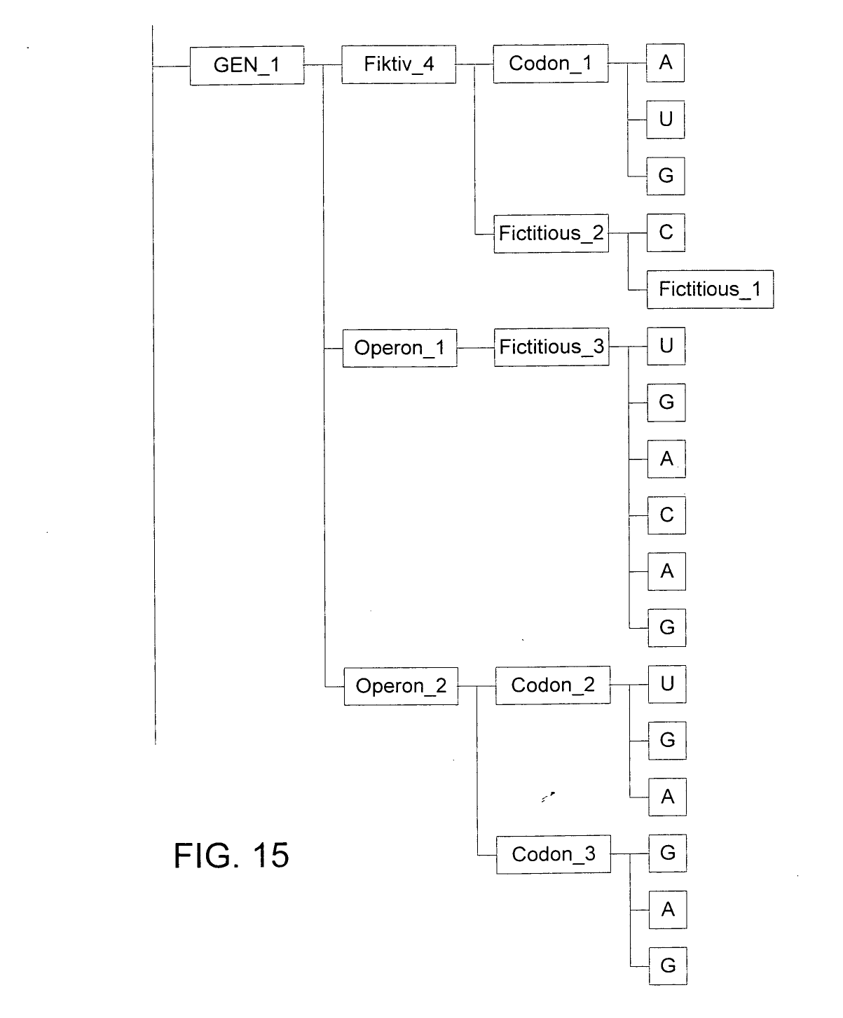

FIG. 15 is a representation of class objects in one grouping hierarchy in accordance with the DNA data structure in FIG. 14;

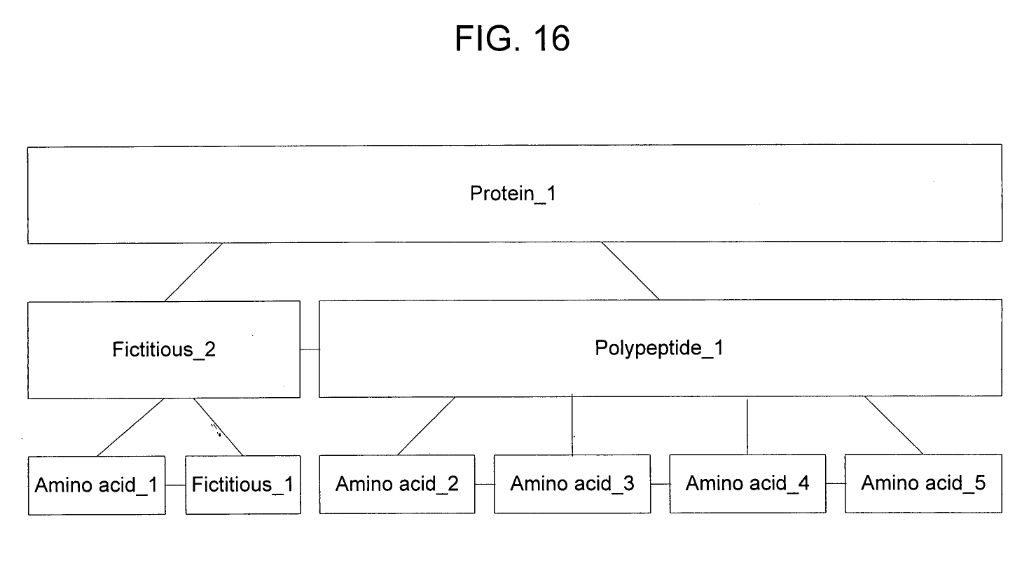

FIG. 16 shows an example of a classified hierarchical network of structure units of a protein data structure in accordance with the practical example of the present invention; and

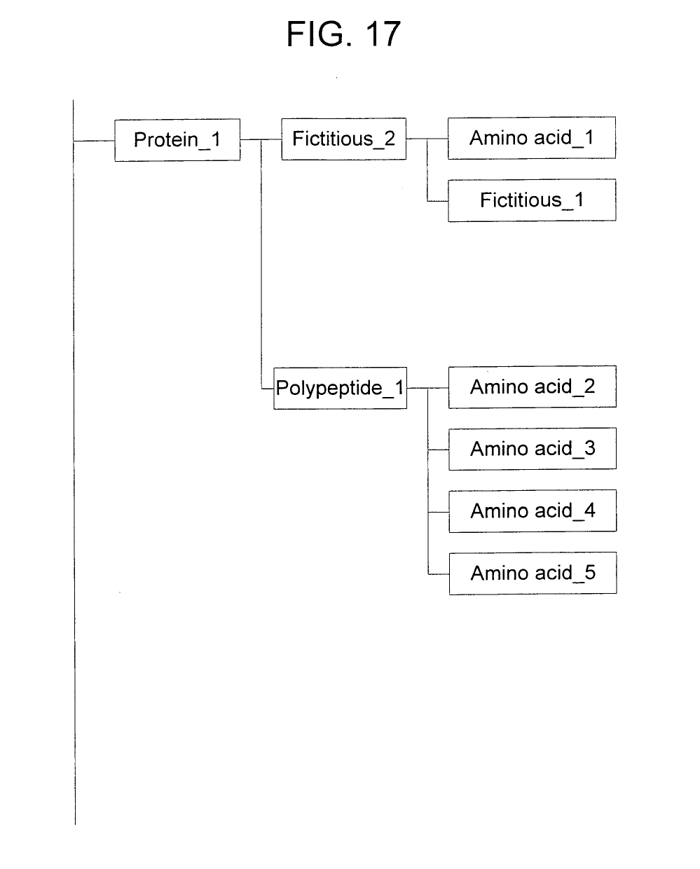

FIG. 17 is a representation of class objects in one grouping hierarchy in accordance with the protein data structure in FIG. 16.

The following is a description of a practical example of the present invention.

With regard to the expressions “semantic network”, “semantic unit”, “linking object” and “processing object” as used in this application, reference is made to the present applicant's application having serial no. 199 60 372.3 and entitled “Verfahren zur Verarbeitung von Datenstrukturen” [Method for processing data structures], filed on Dec. 14, 1999, to the same applicant's application having serial no. 199 08 204.9 and entitled “Fraktales Netz n-ter Ordnung zum Behandeln komplexer Strukturen” [n^{th}-Order fractal network for handling complex structures], filed on Feb. 25, 1999, and the application by the applicant of the present invention bearing serial no. 199 175 92.6 and entitled “Situationsabhängig operierendes semantisches Netz n-ter Ordnung” [nth-Order semantic network allowing for situation-dependent operation], filed on Apr. 19, 1999, wherein the expressions “semantic network” and “fractal network” are to be considered equivalent, the expressions “Janus unit” and “processing object” are to be considered equivalent, and the expressions “linking unit” and “linking object” are to be considered equivalent. The features named in the above specified applications with regard to the structure and the operation of the “fractal network”, of the “semantic network”, of the “semantic unit”, of the “linking unit” and of the “Janus unit” are to be considered included in the present application by reference.

In advance it is noted that the method described hereinbelow may be implemented both on a single computer and on a distributed network of computers, such as a LAN or WAN, wherein the constituents of the semantic network may in the latter case of the network of computers be present both in a centralized and in a decentralized configuration. The method described hereinbelow may thus generally be referred to as a computer-implemented method for processing several data structures.

It is further noted that—although a practical example of the present invention described hereinbelow describes a method for processing genetic data structures of identical or different contents—the present invention is not restricted to this range of application. Rather, the method of the invention may be applied to any data structures that are placed in a context.

Before giving a detailed explanation of the practical example of the present invention, an overview of the structure of the genetic code, of the problems in deciphering the genetic code, and of the mechanism of protein synthesis will now be given.

In accordance with hitherto existing knowledge, the structure of the genetic code is paralleled in linguistics according to the following schema: bases or nucleotides like letters, codogens and codons like words, genes like sentences, operons like sections, chromosomes like documents, DNA and RNA like libraries, knowledge about the genome like the totality of documents containing human knowledge.

Respective definitions of various expressions used hereinbelow are as follows:

Bases are constituents of monomer building blocks of the nucleic acids DNA and RNA, i.e., adenine or A, guanine or G, cytosine or C, and thymine or T (DNA), or uracil or U (RNA).

Base pairs preferably form of A with T (DNA) or U (RNA) and G with C.

Nucleotides are the monomer building blocks of the nucleic acids DNA and RNA.

Codogens (DNA) and codons (RNA) are sequences of three adjacent nucleotides coding for an amino acid, for “START”, or for “STOP”.

Genes are the units in a DNA double strand of chromosomes containing the information, or building instruction, for a protein molecule.

Operons are the units of jointly regulating genes and comprise an operator gene, a regulator gene, and mostly several structure genes.

Chromosomes are the units of the genotype and comprise a thread-type structure wherein the genetic material of cells is laid down.

DNA or deoxyribonucleic acid is a molecular carrier of genetic information.

RNA or ribonucleic acid is another molecular carrier of genetic information, with RNA being synthesized from DNA and occurring in three different forms, i.e., transfer or t-RNA, messenger or m-RNA, and ribosomal or r-RNA.

Genome is the totality of all genes of a cell, i.e., of an organism.

Genetics is the science of the formation of hereditary characteristics and their transmission in successive generations.

Nucleotide sequences are the writing-type sequences of the nucleotides in DNA and RNA.

Amino acids are the building blocks for the proteins, wherein twenty different amino acids exist which are present as L-stereoisomers. Amino acids all have the same basic structure, however differ in one side chain carrying a specific active group.

Proteins are protein substances which are the most important macromolecules of a living cell. Their basic structure is a polypeptide chain containing between one hundred and several hundred amino acid units. This chain folds up in a characteristic manner, whereby different active groups are taken into close vicinity and may form an active catalytic center such as described, for example, in “Stufen zum Leben” by Manfred Eigen, Piper, München, Zürich, 1987.

Living beings use the DNA as a memory for the genetic material and process this stored information according to the following pattern:

DNA as the legislative, RNA as a message, protein as the executive, and metabolism as a function.

The schema and the detail structures are universal. All living beings use a universal genetic code, a universal biochemical machinery, and macromolecular synthesis products.

From the above quoted literature it may further be taken that the preconditions for using nucleic acids as an information memory are satisfied by the chemistry.

As was described above, nucleic acids have four chemically classified compounds, i.e. two purines A and G and two pyrimidines C and T (DNA) or U (RNA). Linking of the nucleotides into a macromolecule ensures metastable and thus merely temporary cohesion of a message. There exists a specific interaction between one purine and one pyrimidine, respectively, mediated through hydrogen bonds, wherein A binds to T (DNA) or U (RNA) and G binds to C. This is referred to as complementarity of the two respective bases capable of interaction. This only makes sure that a message will be readable at all, and that the temporarily stable message will be preserved unlimited through reproduction. Complementarity moreover provides recognition functions which are employed in control and feedback control functions.

Amino acids are allocated to the 4³=64 possible combinations of three of the four bases by the genetic code. Each codogen or codon is unambiguously associated with an amino acid, whereas, however, in return several codogens or codons may be associated with one amino acid. This results in a redundance which is advantageous for a functional language as represented by the proteins. The information generally flows from the DNA via the RNA to the proteins which effect everything else. There do, however, furthermore exist retrogressively writing enzymes which copy RNA information into a DNA double strand. Information laid down in protein chains can, however, not be colinearly reverted to the RNA.

The following problems surface in deciphering the genetic code.

DNA and RNA are macromolecules in which the entire genetic information is stored. The functional units of heredity, i.e. the genes, effect a formation of features in such a way as to control a synthesis of one kind of macromolecules, the nucleic acids, into another kind of macromolecules, the proteins. Both of these kinds of macromolecules have in common that they constitute unbranched chains of a limited selection of single building blocks. The proteins contain twenty kinds of amino acids, approximately one hundred to three hundred of which form a polypeptide chain. The kind and the order of the amino acids cause the polypeptide chain to spontaneously fold into a functional enzyme or structural protein in an orderly fashion. The nucleic acids contain four kinds of nucleotides or bases. The DNA contains A, T, G and C, and the RNA contains A, U, G, C. A linear, writing-type sequence of the building blocks suggests the possibility of the nucleotide sequence of the genes enciphering the amino acid sequence of the proteins in accordance with some rule. In the most simple case, a group of neighboring nucleotides would symbolize an amino acid, and the groups of neighboring nucleotides would follow each other in the same arrangement as the associated amino acids in the polypeptide chain.

By this approach a central biological problem is reduced to the formal question in what way a writing system having twenty letters is enciphered into a writing system having four letters. In order to decide between the multiplicity of theoretical possibilities, the following questions had to be clarified in the past:

1. Does a topical change in the writing system of the nucleic acid indeed bring about a change of a small segment in the amino acid chain?

Answer: A topical change brings about an exchange of single amino acids. Representation exists from point to point.

2. Is the colinearity condition satisfied, i.e., does a sequence of topical changes in the nucleic acid abc always correspond to a sequence ABC of corresponding changes in the amino acid chain and not ACB or BAC?

Answer: There is no exchange of adjacent amino acids, so that an overlapping code is excluded. Hence there is colinearity between the order of the code words in the nucleic acid and the order of the code words in the protein.

3. Do all code words, i.e., nucleotide groups corresponding to an amino acid, have a same length, and if this is the case, the minimum length of three nucleotides for combinatory reasons, or a greater length?

Answer: Three respective nucleotides code for one amino acid. The following is an example for mutations:

(1) DIE RNA IST AUF DEM WEG VOM GEN ZUR TAT

Eliminating one letter (in IST) at the beginning of the sentence results in garble:

(2) DIE RNA ISA UFD EMW EGV OMG ENZ URT AT . . .

The same is true if a letter is added (in IST):

(3) DIE RNA EIS TAU FDE MWE GVO MGE NZU RTA T . . .

Eliminating and adding in two locations that are not too far apart (in IST) about restitutes the meaning:

(4) DIE RNA EIS AUF DEM WEG VOM GEN ZUR TAT

Another two missing or additional letters equally make the meaning incomprehensible, however three letters missing in a small section (in IST and DEM) permit the sentence to be guessed again:

(5) DIE RNA IAU FDE WEG VOM GEN ZUR TAT.

The same effect ensues in the case of three additional letters at the beginning of the sentence.

4. If, for example, the length of the code words is three nucleotides, does then the nucleotide sequence (abcdefghi), wherein the letters designate the positions in the chain, have to be read strongly overlapping (abc bcd cde . . . ), weakly overlapping (abc cde efg . . . ), or not overlapping (abc def ghi)?

Answer: The nucleotide sequence need not be read overlapping, cf. answers for 1. and 2.

5. Is the beginning of a code word marked, or are borders between the code words determined solely by counting the nucleotides from the beginning?

Answer: The resulting question is whereby a beginning and an end of the portion pertaining to a protein is designated on the m-RNA if it determines several proteins. It was found that the corresponding information is present in particular nucleotide triplets or codons: START, i.e., AUG or GUG (if the same code words appear in the running text, they have a different meaning. The closer surroundings of the code words also play a part in determining their meaning), and STOP, i.e., UAG, UAA, UGA.

6. Does the code know punctuation marks, i.e., are the beginning and the end of the code word sequence coding for the entire polypeptide chain marked?

Answer: cf. Answer for 5.

7. What is the dictionary of code words? Are there plural cases such as several code words for one amino acid (degeneration of the code) or several amino acids for one code word (ambiguity of the code)?

Answer: There is a dictionary for the first, second and third letters. In addition the t-RNA has the ability of inserting an amino acid rest, whereby the functionality of a protein is not disturbed, but is continued. This repair mechanism is, however, not operative a hundred percent. UAG, for example, is unambiguously translated as STOP in the non-permissive normal strain of E. coli. In the permisssive host of E. coli, however, it may be translated both as STOP and as serine, and thus is ambiguous. Ambiguity in reading the m-RNA may also be caused in vitro through change of external factors. Investigation into the role of different kinds of t-RNA in the translation of base triplets of identical meaning showed that although there are more different t-RNA molecules than amino acids, there nevertheless are less t-RNA molecules than base triplets.

The number of kinds of t-RNA therefore is located between the number of the base triplets (64) and the number of the amino acids (20). In many cases a single t-RNA thus has to recognize more than one base triplet. This conclusion is not in harmony with a perfect base pairing between the codons on the m-RNA and the anticodons on the t-RNA. Crick hence proposed a model wherein the base pairing in the third position of the m-RNA codon has a certain play between several possibilities. This is referred to as the wobble hypothesis and at the same time explains several irregularities in the code schema.

8. Closely connected with 7.: Is the code in a particular organism entirely independent of the physiological conditions, and, if this is at least largely true, is the code the same for all organisms, i.e.: is the code universal?

Answer: With the restriction of a suppression, the extreme change of external factors, and certain toxic influences, the code may be considered constant for a given organism. It is therefore sensible to compare the code of different organisms that are distant within the system. As was mentioned above, the code is universal. It is, however, not known whether this is due to the reason that some structural relationship or other between amino acid and anticodon on the t-RNA has to exist, or that mutations bringing about changes of meaning in the code are nearly always lethal because of the effect they have on the entire protein synthesis of the organism, and therefore do not take effect in evolution.

The direct path for answering the above questions would simply consist in the comparison of a known amino acid sequence with the associated nucleotide sequence. Because of the technical difficulties in sequence analysis, however, such a comparison presently still is not possible despite intense efforts. It nevertheless was possible to answer all of the above questions. Execution and interpretation of the decisive experiments for the purpose of answering the above questions hinge on knowledge about the biochemical process of protein synthesis.

The following is a description of the protein synthesis mechanism.

In protein synthesis there are two essential processes, namely, transcription and translation.

In transcription, the genetic information of the DNA in the nucleus is transferred to the m-RNA. This process resembles DNA replication. The two strands of the DNA double strand locally separate from each other, and the bases, now not paired any more, enter into interaction with complementary ribonucleotides which are then linked by an enzyme, i.e., by the RNA polymerase. The polynucleotide thus newly formed detaches from the DNA to migrate as a one-stranded molecule from the nucleus into the cytoplasm. The information contained in the nucleotide sequence of the DNA thus is contained in the nucleotide sequence of the m-RNA and thus is transportable.

During translation in the cytoplasm, ribosomes thread onto the m-RNA. Amino acids are bound to t-RNA in the cytoplasm. These “loaded” t-RNA molecules have the ability of entering into interaction with the m-RNA through three of their bases, i.e., the anticodon, and thus can read the code contained in the codon. To be more precise, the amino acids bound to the t-RNA molecules are linked into polypeptide chains at the ribosomes. The t-RNA molecules thus have the function of an interpreter translating the language of the DNA, i.e., a sequence of bases, into the language of the proteins, namely, a sequence of amino acids.

The following is a description of the structure and function of the practical example of the present invention.

In the framework of the invention it was recognized that in many applications a meaningful description of objects in data structures, i.e., the grouping of single data, often is very difficult to perform automatically. The single data may be heterogeneous, very sophisticated semantics may be required in order to describe what single data of a data structure pertain to a particular object, and/or criteria permitting the extraction of objects differ between different object classes in a same data structure. Through the method described in the following there is created, for example, a possibility of extrapolating which permits the information not directly contained in acquired data to be supplemented.

In addition, much more voluminous information is used for making decisions than only information reproducible by itself through a structure object. Rather, very often it even only is the relations or relationships that enable a decision. Such a relationship may, for example, be an embedding in particular environs, a positional relation, the relationship of certain properties of an object with other objects, or a composition of particular partial areas.

Accordingly in the method described in the following, processing steps take place not solely by global criteria but are also carried out specifically and locally. Local processing is here made possible by structure objects, i.e., the object-type representation of an area of the data structures.

It is only through the intermediary of such structure objects substitutionarily representing quite a particular area of the data structures, that decisions and processing may be carried out locally in a specific manner. Areas may, for example, be taken into a mutual relationship by comparing properties of respective structure objects. Finally it is possible to operate locally and specifically through a classification of structure objects.

The above explanations become apparent more clearly through the following detailed description of the practical examples of the present invention.

One essential feature of the method described in the following is the formally independent consideration of a network that consists of semantic knowledge units and contains the knowledge base necessary for analysis and processing, and of hierarchical networks of semantic structure units wherein the information (single data) contained in the data structures to be processed is at a same time present at various degrees of resolution and moreover as a network.

For processing the information contained in data structures, the networks of semantic structure units and the network of semantic knowledge units are again and again interlaced with each other in an identical or different manner. In order to achieve a relationship between various data structures, different data structures pertaining to different networks of semantic structure units are moreover interlaced with the aid of a coupling network.

One essential feature of the method described hereinbelow therefore is the kind of semantic units of which the semantic networks are constructed, the corresponding network structures, and the manner in which information has to be processed so that the like networks and the units contained in them may be generated, changed, and used in terms of information.

For the purpose of elucidating the structure of the semantic networks employed in the present application, in the following initially an explanation of the “building blocks” of a semantic network, the semantic units, is given. Semantic units serve the purpose of receiving informational contents, with six different basic types of semantic units existing in the present method, namely, structure objects, linking objects, class objects, analysis objects, processing objects and feature objects.

Structure objects are either each unambiguously tied in with a specific area of a data structure and represent it and/or properties thereof, or they are not, or not exclusively, tied in with a specific area of the data structure but supplement information not directly contained in a data structure.

Linking objects each link two arbitrary semantic units among each other in such a way that the kind and the informational content of a respective linking object determine the relationship of the two semantic units among each other.

Class objects represent a class and in particular apply analysis objects and/or processing objects linked with them to semantic units. With the aid of the class objects, semantic units may be analyzed, classified, differentiated, allocated to a class, and/or either themselves or particular partial networks may be modified. A class object may also transfer attributes to semantic units allocated thereto. Class objects constitute the basic unit for the cycle of “analysisdecision-processing” recurringly taking place.

Through linking with feature objects, analysis objects include evaluation algorithms whereby they analyze those semantic units to which they are linked, and/or particular partial networks, e.g. the respective networked environs. Analysis objects are in particular tied in with class objects and are applied to those semantic units to which the respective class object is analyzingly connected.

Processing objects each contain processing algorithms whereby they modify those semantic units to which they are linked and/or particular partial networks, for example the respective networked environs, as well as sequencing controls with respect to these algorithms. Processing objects are in particular tied in with class objects and are applied to those semantic units with which the respective class object is connected in a processing manner or possibly in an allocating manner.

Feature objects contain feature descriptions and evaluation algorithms for the purposes of local analyses. The like features and evaluation algorithms contain attributes or templates, for instance.

Semantic units include an informational content. This informational content is classified into the three ranges of designation or unambiguous addressing (ID), of data and functions of the semantic unit (DF), and enumeration of all linking objects connecting a respective semantic unit with other semantic units. The enumeration of the linking objects in a semantic unit may, for example, also be present in the form of a classification, so that the relationships realized by way of linking objects are ordered by contents.

The term “semantic structure units” subsumes structure objects, their links among each other, and networks/partial networks of semantic structure units. The term “semantic knowledge units” subsumes feature objects, analysis objects, processing objects, class objects, and their links among each other.

One essential term is the concept of a “particular partial network”, presently and in the following designating all further semantic structure units of a particular type and/or of a particular classification at a particular distance which, starting out from one of a plurality of particular semantic structure units, are linked therewith either directly or indirectly via other semantic units. A “particular partial network” may in particular also be the networked environs of a semantic structure unit.

A first semantic unit is, for example, defined as pertaining to networked environs of a second semantic unit when a distance between these is smaller than a predetermined or calculated value, i.e. a limit value. A measure of the distance depends on informational and/or connotational contents of the semantic units through which the second semantic unit may be reached from the first semantic unit. It is, for example, possible to calculate the measure of the distance through weightings in linking objects, wherein it is possible for the type of the linking object to also enter into this calculation.

Such weighted linking is, for example, realized by entering a weighting into the informational content of a semantic unit. The networked environs are then defined to the effect that—when starting out from one semantic unit—all those other semantic units directly or indirectly linked with this semantic unit pertain to the networked environs of this semantic unit which may be reached along the linking path by continuous multiplication of the weightings without exceeding or dropping below a predetermined threshold.

As was already mentioned above, linking objects link semantic units among each other. Linking objects are an important precondition for modelling and processing image semantics. In the most simple case, a linking object is a designation or ID, a pointer or a logic link.

The nature of a linking object is essentially responsible for the quality of a linking. The nature of a linking object is determined by a designation of the linking object in the respective informational content and/or by the location or the enumeration in a semantic unit with which the linking object is connected. Particular types of linking objects characterize a respective partial network or a partial space in a hierarchically structured fractal network.

The relationship of two semantic units realized by linking objects need not be symmetrical, i.e. ambiguous in either direction like, for instance, a relationship “is a neighbor of”, but in most cases will rather be unsymmetrical, i.e., unambiguous in both directions, like for example a relationship “is sub-object of” and “is super-object of”.

It should furthermore be pointed out that linking objects may in turn be linked through further linking objects. This results in a general fractal structure of the network. In detail, semantic units may thus be linked with semantic units via linking objects, semantic units may be linked with linking objects via linking objects, and linking objects may be linked with linking objects via linking objects. This results in a fractal structure of a semantic network.

The most important types of linking objects shall in the following be described in more detail.

A linking object of type VS links structure objects with each other.

A linking object of type VSH hierarchically links structure objects with each other and constitutes an unsymmetrical linking which differentiates between super-structure objects and sub-structure objects.

A linking object of type VSN links neighboring structure objects with each other and constitutes a symmetrical linking. The expression “neighboring” here refers to structure objects of a same plane of hierarchy in the hierarchical network of structure objects.

A linking object of type VK constitutes a class link which links a semantic unit with a class object.

A linking object of type VKA constitutes a classifying link applying an evaluation algorithm contained in an analysis object linked with a respective class object to semantic structure units and/or particular partial networks.

A linking object of type VKZ constitutes an allocating class link which allocates a semantic unit to a class object and thus to the class represented by the class object. Its meaning may be expressed as “in general is” and “in particular is”, as a result of which it is synonymous with a linking object of type VSH already explained above. A linking object of type VKZ can transfer attributes from the respective class object to the linked semantic unit.

A linking object of type VKP constitutes a processing class link that applies a processing algorithm contained in a processing object linked with a respective class object to the semantic unit and/or particular partial networks.

A linking object of type VÄ constitutes a similarity link that links class objects with each other along a similarity of feature descriptions with regard to analysis objects, or of processing algorithms with regard to processing objects, whereby a similarity hierarchy of class objects is generated.

A linking object of type VÄH constitutes a hierarchical similarity link that hierarchically links class objects with each other and passes on links with analysis objects, attributes and/or processing objects from super-class objects to sub-class objects by heredity.

A linking object of type VÄHA constitutes a hierarchical similarity link with regard to feature descriptions concerning analysis objects, that passes on links with analysis objects from super-class objects to sub-class objects by heredity.

A linking object of type VÄHJ constitutes a hierarchical similarity link with regard to processing algorithms concerning processing objects, that passes on links with processing objects from super-class objects to sub-class objects by heredity.

A linking object of type VÄN constitutes a neighborhood similarity link that in particular links such class objects with each other that are connected with the same super-class object via a respective linking object of type VÄH, and thus inherit identical feature descriptions with regard to analysis objects, attributes and/or processing algorithms concerning processing objects, and in this respect are considered to be similar.

A linking object of type VÄNA constitutes a neighborhood similarity link with regard to feature descriptions concerning analysis objects.

A linking object of type VÄNJ constitutes a neighborhood similarity link with regard to processing algorithms concerning processing objects.

A linking object of type VG constitutes a grouping link that links class objects with each other with regard to their semantic meaning and groups them, whereby a connotation hierarchy or grouping hierarchy of class objects is generated.

A linking object of type VGH constitutes a hierarchical grouping link which hierarchically links class objects among each other, with the semantic meaning being “in general is” in an upward direction in the hierarchy, and “in particular is” in a downward direction in the hierarchy. This linking object is related to a linking object of type VKZ to the effect that instead of a structure object, as is the case with the linking object of type VKZ, one class object is here allocated to another class object as a sub-class object.

A linking object of type VGN constitutes a neighborhood grouping link which links class objects of a similar semantic meaning with each other in a neighborhood manner, i.e. on a same hierarchy level.

A linking object of type VM constitutes a feature link which links analysis objects and feature objects among each other and applies feature descriptions contained in the feature objects and evaluation algorithms to the semantic structure units that are connected with a respective analysis object.

A linking object of the type VÄG represents a grouping similarity link having the properties and functions both of the linking object of type VÄ and of the linking object of type VG.

A linking object of type VÄGH constitutes a hierarchical grouping similarity link which exhibits the properties and functions both of the linking object of type VÄG and of the linking object of type VGH.

A linking object of type VÄGHA constitutes a hierarchical grouping similarity link which exhibits the properties and functions both of the linking object of type VÄHA and of the linking object of type VGH.

A linking object of type VÄGHJ constitutes a hierarchical grouping similarity link which exhibits the properties and functions both of the linking object of type VÄHA and of the linking object of type VGH.

A linking object of type VÄGN constitutes a neighborhood grouping similarity link which exhibits the properties and functions both of the linking object of type VÄN and also of the linking object of type VGN.

In the following, a description of the hierarchical semantic networks of structure units will be given by way of the above described structure objects and their links.

FIG. 1 shows a schematic representation of several coupled hierarchical networks of structure units in accordance with an embodiment of the present invention.

As was mentioned above, in the practical example of the present invention several different data structures are processed that are related to each other, i.e. that have relationships among each other. In the case of this practical example, data structures relating to DNA, RNA and to proteins are used.

At first, however, the structure of the coupled hierarchical networks of structure units shall here be explained in general.

In FIG. 1, reference numeral 1 designates the lowermost hierarchy level of respective semantic networks of structure units, reference numerals 2, 3 and 4 designate different hierarchy levels of the hierarchical semantic networks of structure units, reference numeral 5 designates an exemplary linking object of the type VSN, reference numeral 6 designates an exemplary linking object of the type VSH, reference numeral 7 exemplarily designates a data point object, and reference numeral 8 exemplarily designates structure objects.

As was already mentioned above, structure objects 8 are each tied in with an area of the data structures to be processed while representing it. Any single point of the data structures to be processed may be represented by a structure object 8 or in the hierarchical sense by several structure objects 8. The structure objects 8 may be coherent with regard to the topology of the acquired data structure, however do not of necessity have to.

The structure objects 8 contain data describing the individual properties of a respective structure object 8 such as, e.g., size, position, etc.

Further information about the context is contained in the enumeration of linking objects 5 and 6 connected with structure object 8, and in the informational content of structure object 8. The enumeration is present in a form that is structured in accordance with sub-structure objects, super-structure objects, i.e. linked through a linking object 6 of type VSH, and neighbor-structure objects, i.e., linked through a linking object 5 of type VSN.

This can be seen in FIG. 1. For example in the topmost hierarchy level 4 of the hierarchical semantic networks of structure units, two structure objects 8 are linked to each other by a linking object 5 of type VSN miteinander and accordingly are in a neighborhood relation. Moreover, for example, a structure object 8 is linked in the uppermost hierarchy level 4 of the hierarchical semantic networks of structure units via a linking object 6 of type VSH with a structure object 8 in the next-lower hierarchy level 3 of the hierarchical semantic network of structure units, which means that the structure object 8 in the uppermost hierarchy level 4 of the hierarchical semantic network of structure units is a super-structure object of the structure object 8 in the next-lower hierarchy level 3 of the hierarchical networks of structure units, and vice versa the structure object 8 in the next-lower hierarchy level 3 of the hierarchical semantic network of structure units is a sub-structure object of the structure objects 8 in the uppermost hierarchy level 3 of the hierarchical network of structure units. As may be seen in FIG. 1, a multitude of such linkings are present in such hierarchical semantic networks of structure units by way of linking objects 5 of type VSN and linking objects 6 of type VSH.

Another component of linking objects 5, 6 of type VS may moreover be constituted by data designating properties of a respective link, such as, e.g., the length of a common edge of neighbor-structure objects in the case of a linking object of type VSN.

It is noted that in the above described structure of the hierarchical networks of structure units, different hierarchy levels 1 to 4 may be formed, as is represented in FIG. 1, and by means of these different hierarchy levels 1 to 4 of the structure objects 8 the contents of data structures to be processed may be represented at various degrees of resolution at the same time. This results in the hierarchy of the semantic network in conformity with topological positional relations of the data structure to be processed.

Such hierarchical networks of structure units need not have a unified hierarchical depth, i.e., an identical number of hierarchy levels 1 to 4 in any location, and it is not always necessary for linking objects 5 of type VSN to be contained in the semantic networks of structure units. Furthermore it is not always necessary for the semantic networks to be hierarchically unambiguous. This means that there is a possibility of a sub-structure object having several super-structure objects.

It is noted, however, that with the exception of the data point object 7 a structure object 8 always represents the one area of the data structure to be processed which is composed of the entirety of all areas represented by the respective sub-structure objects of this structure objects 8. In this regard, the hierarchical semantic networks of structure units are unambiguous in accordance with the topology of the data structures to be processed.

Although in FIG. 1 three networks of semantic structure units, which have a same structure, are shown for the sake of simplicity, it is self-evident that the several semantic networks of structure units wherein several different data structures are represented, as a rule have different structures in accordance with contents of the respective data structures. In order to incorporate a connection between the respective data structures in processing, the semantic networks of structure units are networked with each other via a coupling network, as is indicated in FIG. 1 with the aid of the three double arrows. Such networkings between the semantic networks of structure units may act in both directions.

By referring to FIGS. 2 to 10, in the further continuation local operations shall be described which may be carried out within the hierarchical semantic networks of structure units.

Although several examples on particular hierarchy levels of the semantic networks of structure units are given hereinbelow, it is noted that these local operations may take place both between all hierarchy levels and between the several networks of semantic structure units themselves, i.e., within the coupling network.

In the following a structure of three networks of semantic structure units processing genetic data structures is assumed. To be more precise, a DNA network of semantic structure units, an RNA network of semantic structure units, and a protein network of semantic structure units are assumed, each one of which is interlaced with the network of semantic knowledge units. Between these three networks of semantic structure units the coupling network is formed in order to allow for processings between the several different data structures, namely, DNA, RNA and protein data structures as well as their respective super-objects and sub-objects.

The hierarchy in the DNA and RNA network of semantic structure units from the bottommost hierarchy level to the uppermost hierarchy level is as follows, for example: nucleotides, codogens (DNA) or codons (RNA), i.e., nucleotide triplets, groups of codogens or codons and their relationships among each other, operons, groups of operons and their relationships among each other, chromosomes, groups of chromosomes and their relationships among each other, genes and groups of genes and their relationships among each other, DNAs or RNAs, groups of DNAs or RNAs of a kind and their relationships among each other, genomes, groups of genomes and their relationships among each other. Additional structure units are the relationships or linking objects which link structure units on the same hierarchy level as neighbor objects, and structure units on different hierarchy levels as sub-objects or super-objects.

The hierarchy in the protein network of semantic structure units from the bottommost hierarchy level to the uppermost hierarchy level, for example, is as follows: amino acids, groups of amino acids and their relationships among each other and proteins and groups of proteins and their relationships among each other. Additional structure units are the relationships or linking objects, which link structure units on the same hierarchy level as neighbor objects, and structure units on different hierarchy levels as sub-objects or super-objects.

It is important that the size or order and the position within an array of objects plays an important part in what properties a respective object has, as will be seen more clearly hereinbelow.

FIG. 2 shows a local “fusion” operation within the hierarchical networks of semantic structure units with the aid of a respective network section in accordance with the practical example of the present invention.

In the case of the local “fusion” operation, two or more already existing structure objects 8 shown in the left-hand representation of FIG. 2 are fusioned or melted into a new structure object 8, as can be seen in the right-hand representation of FIG. 2.

In FIG. 2 the lower hierarchy level is the hierarchy level of codons, and the upper hierarchy level is the level of genes. Thus codons a, b and c and genes a and bc are represented in the left-hand representation of FIG. 2. In FIG. 2, the gene a and bc fuse with each other to form a new gene of the three codons a, b and c. Here the order of the codons in the newly formed gene plays an important part. From top to bottom on the right-hand side in FIG. 2, four such examples are represented:

Gene abc: codons a, b and c is not

Gene bac: condons b, a and c is not

Gene bca: codons b, c and a is not

Gene cba: codons c, b and a is not

etc. for any possible combinations.

Neighborhood within a hierarchy level is defined by an order within a neighborhood list.

In the following another example of the local “fusion” operation on other hierarchy levels is given:

Codon abc: nucleotides a, b and c is not

Codon bac: nucleotides b, a and c is not

Codon bca: nucleotides b, c and a is not

Codon cba: nucleotides c, b und a is not

etc. for any possible combinations.

Thus it may be seen that the order of the nucleotides, i.e., of the sub-objects, within a list in one group, or of a codon, i.e. of the super-objects, determines an identity of a respective super-object. This order equally takes effect between the respective hierarchical networks of semantic structure units and takes effect, for example, as far as into the protein network of semantic structure units in protein synthesis. The effect thus goes beyond the own data structure.

As can be seen from the above explanations, the order of the sub-objects substantially determines the order of the super-objects and thus the identity of the entire data structure. This is true for all of the local operations described hereinbelow.

FIG. 3 shows a local “founding” operation within the hierarchical network of semantic structure units with the aid of a respective network section in accordance with the practical example of the present invention.

In the local “founding” operation, a new structure object 8 is generated as a new super-structure object for one or several structure objects 8 shown in the left-hand representation of FIG. 3, and linked with these via linking objects 5 of type VSH, as may be seen from the right-hand representations of FIG. 3.

From top to bottom on the right-hand side in FIG. 3, four such examples are represented:

Codon a, b and c found gene abc or

gene bac or

gene bac or

etc. for any possible combinations.

Here it may also be seen that the order of the sub-objects, i.e., codons, within a list of the sub-objects substantially contributes to the identity of the created gene.

FIG. 4 shows a local operation “insertion of a neighbor as a sub-structure object” within the hierarchical networks of semantic structure units with the aid of a respective network section in accordance with the practical example of the present invention.

In the local operation “insertion of a neighbor as a sub-structure object”, an existing linking object 5 of type VSN between neighboring structure objects 8 is dissolved and a new linking object 6 of type VSH is created between them, whereby a previous neighbor-structure object is allocated to structure object 8 as a sub-structure object, and correspondingly the area of the data structure to be processed and pertaining to structure object 8 increases in size.

As is represented in FIG. 4, for example during a classification a gene 2 meets a codon 1. In the process the codon 1 is subordinated to the gene 2 as a sub-structure object. The properties and the identity of the created super- or sub-object are for the most part determined by the position of the sub-object within the super-object, as is indicated by the dashed positions of the codon 1 in the gene 2. If the sub-object is located in the first, second, third, etc. position of the super-object, then entirely different super-objects with different properties and different identities are created which take effect acrross the entire networks of semantic structure units, i.e. via DNA to RNA and proteins and vice versa.

In the other example represented in FIG. 4, gene 2 meets a group of codons abc during a classification. In the process, the gene 2 is subordinated to the group of codons abc as a sub-structure object and furthermore the group of codons abc is classified to gene 2, whereby a new super-structure object results from gene 2 and the group of codons abc.

FIG. 5 shows a local “exclusion” operation within the hierarchical network of semantic structure units with the aid of a respective network section in accordance with the practical example of the present invention.

In the local “exclusion” operation, an existing linking object 6 of type VSH between sub-structure object and a super-structure object is dissolved, whereby the previous sub-structure object is excluded, and correspondingly the area pertaining to structure object 8 of the data structure to be processed is reduced in size.

As is shown in FIG. 5, a gene abcacb includes the codons a, b, c, a, c and b as sub-structure objects. From this group of codons any codon may be excluded. What gene results as a super-structure object after the exclusion depends on which one of the codons is excluded. Entire groups of sub-structure objects may equally be excluded, wherein the position of the group of codons within a respective gene also is of importance.

In FIG. 5 in the first case the first codon a is excluded from the gene abcacb, and following the exclusion is located in front of the codons pertaining to the gene as sub-structure objects, so that the gene bcacb results as a new super-structure object.

In FIG. 5 in the second case the first codon a is excluded from the gene abcacb, and following the exclusion is located behind the codons pertaining to the gene as sub-structure objects, so that the gene bcacb results as a new super-structure object.

In FIG. 5 in the third case the second codon b is excluded from the gene abcacb, and following the exclusion is located in front of the codons pertaining to the gene as sub-structure objects, so that the gene acacb results as a new super-structure object.

In FIG. 5 in the fourth case the second codon b is excluded from the gene abcacb, and following the exclusion is located behind the codons pertaining to the gene as sub-structure objects, so that the gene acacb results as a new super-structure object.

Apart from the examples shown in FIG. 5, any other possibilities are conceivable.

FIG. 6 shows a local operation “insertion of a new sub-structure object” within the hierarchical network of semantic structure units with the aid of a respective network section in accordance with the practical example of the present invention.

In the local operation “insertion of a new sub-structure object” a new linking object 6 of type VSH is generated between a structure object 8 and another structure object 8 which hitherto was not yet a super-structure object of this structure object 8, whereby a new sub-structure object is allocated to the super-structure object. and correspondingly the area pertaining to the super-structure object of the data structure to be processed is increased in size.

As is shown in FIG. 6, a gene abc includes the codons a, b and c as sub-structure objects. To this gene abc another codon a is associated, resulting, for example, in the genes abca, aabc, abac, but with any further combinations also being conceivable.

FIG. 7 shows a local “dividing” operation within the hierarchical networks of semantic structure units with the aid of a respective network section in accordance with the practical example of the present invention.

In the local “dividing” operation, a structure object 8 is divided into several new sub-structure objects 8 on the basis of its sub-structure objects. This means that sub-structure objects are grouped into two or more areas of the data structure to be processed, which are each represented by a structure object 8 to be newly generated on the same hierarchy level of the hierarchical network of structure units as the divided structure object 8.

As is shown in FIG. 7, the gene abbcabca . . . is divided into the three new genes abb, cab and ca . . . which are located on a same hierarchy level as the original gene abbcabca . . . . Here again any possibilities are conceivable.

FIG. 8 shows a local “regrouping” operation within the hierarchical networks of semantic structure units with the aid of a respective network section in accordance with the practical example of the present invention.

In the local “regrouping” operation, at first the local “exclusion” operation described by referring to FIG. 5, and then the local operation “insertion of a new sub-structure object” described above by referring to FIG. 6 takes place.

As is shown in FIG. 8, for example a codon c is excluded from the gene abc and inserted into the gene acb, resulting, e.g., in the new genes ab and accb or ba and acbc. Moreover any further possibilities are conceivable.

FIG. 9 shows a local operation “jumping/exchanging” within the hierarchical networks of semantic structure units with the aid of a respective network section in accordance with the practical example of the present invention.

In the local “jump” operation, the positions of sub-structure objects of a super-structure object are replaced, which means that one or several sub-structure objects jump from one position to another position.

As is shown in FIG. 9, for example the codons a and b change positions within the gene abc, whereby the new gene bac is created. Such changing may, for example, have some context-related reasons. By exchanging a mutation is caused.

FIG. 10 shows a local “boundary optimization” operation within the hierarchical networks of structure units with the aid of a respective network section in accordance with the practical example of the present invention.

In the local “boundary optimization” operation, marginal sub-structure objects of a structure object 8 are analyzed to the effect whether or not they better match a neighbor-structure object of the previous super-structure object than with the previous super-structure object in terms of a predetermined criterion. If they better match a neighbor-structure object, a linking object 6 of type VSH with the previous super-structure object of this marginal sub-structure object is obliterated and a new linking object 6 of type VSH with the better matching structure object 8 is generated to thereby become the new super-structure object.

In FIG. 10 the boundary optimization is carried out such that structure objects designated as abc, dc, bac and aa are boundary optimized. To be more precise, the structure objects designated as d and c, which are sub-structure objects of the structure objects 8 designated as abc bzw. bac, are boundary optimized in such a way that the structure object 8 designated by ddc is allocated to the structure object 8 designated by d as a new super-structure object, and the structure object 8 designated by caa is allocated to the structure object 8 designated by c.

Besides the local operations described above and shown in FIGS. 2 to 10, the following local operations may moreover be performed within the semantic networks of structure units.

In a local “copy” operation, an object is created as a copy of another object. For example a m-RNA sequence UAC is created from a DNA sequence ATG by copying information and applying processings corresponding to the above described genetic mechanism.

In a local “docking” operation, a neighborhood is formed between objects in different networks of semantic structure units. For example in protein synthesis, only particular combinations of nucleotides in the t-RNA and thus amino acids are docked to particular combinations of nucleotides in the m-RNA.

This means that a certaom object from a first network of semantic structure units may only admit a neighborhood with a particular other object from a second network of semantic structure units. The neighborhood only forms for a particular duration of time. This has the purpose of information transfer. The neighborhood redissolves once the purpose of the information flow has been fulfilled.

In all of the above described local operations, the positions and the sizes of respective objects are essential for identities and effects with respect to other objects and the range of distance of these effects.

The coupling network between the networks of semantic structure units containing different data structures uses all of the above described processings in accordance with demand and is constructed in accordance with the same principle as the networks of semantic structure units for the different data structures.

Evidently the above processings may take place on any possible hierarchy levels and between the respective networks of semantic structure units. For example what is true for genomes may quite the same apply to peptides or proteins or other networks which are not necessarily neighboring. In the same way, in order to be able to draw conclusions back to such processings, it iis possible to underlie for the entire organism a corresponding description which may serve a further step, such as diagnostic purposes, for example. For example genetic reasons may be detected and pursued better then previously. Man, or generally any one or several organisms may thus be construed as a network of mutually networked data structures of different contents. This has the decisive advantage that information flows and quality leaps within and without the system may be pursued in all directions, even historically.

In the following the description of the network of semantic knowledge units is given with the aid of the above described class objects, analysis objects, processing objects and their linkings, as well as a representation of the analysis and processing algorithms.

The class objects contained in the network of semantic knowledge units and described above have three different basic functions.

On the one hand, they act analytically, which means that semantic units and/or particular partial networks, in particular the respective networked environs, are analyzed with the aid of analysis objects linked with a class object. This is in particular performed with regard to a pertinence to the class represented by the class object and with the aid of the above described linking object of type VKA.

They moreover act allocatingly, which means that semantic units may be linked with a class object, preferably on the basis of a preceding analysis, by the above described linking object of type VKZ, and thus be associated to the class represented by the class object.

Finally they act processingly, which means that semantic units are linked by the above described linking object of type VKZ with a class object, and a processing algorithm contained in a processing object pertaining to this class object is applied to the semantic structure units and/or particular partial networks via a linking object of type VKP and the class object.

It is noted that in this method not only structure units, i.e., structure objects and/or their links, are classified with the aid of class objects. There is also a possibility of classifying any kinds of semantic units, which in particular applies to linking objects or class objects. As a result of this possibility of classifying any semantic units, the possibility of describing contents of the data structures to be described and to be processed is increased considerably.

Class objects, in turn, may in particular themselves be part of analysis algorithms of a feature object, for example for the extraction of a partial network for a particular analysis, or part of processing algorithms in processing objects, for example for performing a particular step requiring additional analysis, in the entire process described by the processing object. Due to the fact that feature objects and processing objects themselves are constituents of class objects, there results in this way a fractal-hierarchical structure of class objects in the network of semantic knowledge units, bringing about a corresponding fractal-hierarchical structure of analyzing and/or processing steps.

As was described above, analysis objects include evaluation algorithms with the aid of which they analyze semantic units linked with them, and/or particular partial networks. Analysis objects are in general linked with a class object and are applied to those semantic units with which they are analyzingly linked, and/or to particular partial networks.

The above described evaluation algorithm may be an enumeration of criteria, with the aid of which a degree of a pertinence of semantic units to the class object linked with an analysis object is determined. Such criteria are defined with the aid of feature objects, wherein the feature objects are connected with a respective analysis object via the linking object of type VM.

Each feature object evaluates a respective one of the features described hereinbelow. The feature objects are applied singly or in groups, and the results of all evaluations carried out are linked with each other through a logic or a logic link.

This logic may generally be predetermined, such as for example averaging of the results of all criteria, classified hierarchically, indicated specifically for a respective group of feature objects, or formulated with the aid of a fuzzy logic.

The linking objects of type VM may moreover be weighted, with the weighting of this linking object of type VM accordingly yielding the measure whereby the respective feature object, or the feature contained therein, is taken into account in an overall evaluation.

With the aid of class objects contained in them and the processing objects thereof, the feature objects may perform virtual or merely temporary structural modifications within the semantic network, in order to evaluate a structural modification within the semantic network potentially following from a respective classification in terms of its result, and to thus classify the semantic unit in question accordingly. By with the aid of the processing objects it is furthermore possible to carry out actual modifications that are required for performing a local decision with regard to a classification. For example, with the aid of the processing objects it is thus possible, for the purpose of a further differentiation of structure objects, to form sub-structure objects are formed; through the intermediary of their classification and composition, the structure objects to be actually classified are then differentiated.

Features contained in feature objects and intended to serve for evaluation may be features that relate to the property of semantic units, without the analysis of other semantic units or the relationships with them, features resulting from an analysis of a property of the networked environs of a semantic unit, for example a composition of sub-structure objects with the aid of a classification; features resulting from a comparison of a property of a semantic unit with a corresponding property of its networked environs; and features resulting from a comparison of a property of a semantic unit with a corresponding property of a particular partial network. It may, however, for example also be a matching of a structure object with a predetermined template or other analyses

Features contained in feature objects may furthermore result from an analysis of the classification of semantic units of a particular partial network.

As was described above, the feature objects are used for describing and evaluating single features or attributes, respectively.

The evaluation algorithms contained in them may be of various kinds, such as fuzzy pertinence functions, classifiers evaluating by way of particular “training objects” with the aid of a Nearest-Neighbor method, neuronal networks, statistical analyses or shape templates, whereby semantic units or partial networks of semantic units to be classified or optimized are compared.

Feature objects may be linked both with analysis objects and also with processing objects via the linking object of type VM and are applied by these to the respective semantic units and/or particular partial networks to be processed.

In accordance with the above description, processing objects contain processing algorithms whereby they modify the semantic units and/or particular partial networks directly or indirectly linked with them via a class object. Such modifications are in particular any local operations as already described with regard to the hierarchical network of structure units, wherein instead of the structure objects and their links in general, all kinds of semantic units and links thereof may be processed by means of the local operations; this means that in particular semantic units may be deleted, generated, modified, or linked with already existing semantic units in the hierarchical semantic network.

In order to support a processing, processing objects may carry out partial steps requiring an additional analysis with the aid of class objects linked with them, even during ongoing processing.

For the purpose of structuring and reduction of complexity of “world knowledge”, class objects may moreover be linked among each other via different linking objects.

These are the above described linking objects of type VÄ with the subordinate linking objects of types VÄH, VÄHA and VÄHJ, VÄN, VÄNA and VÄNJ, linking objects of type VG with the subordinate linking objects of types VGH and VGN, as well as the linking objects of type VÄG with the subordinate linking objects of types VÄGH, VÄGHA, VÄGHJ and VÄGN.

Herein the linking object of type VG and its subordinate linking objects of types VGH and VGN, which constitute a grouping linking, create a relationship having the meaning “is (semantically) in general” in an upward direction within the hierarchical structure and the meaning “is (semantically) in particular” in a downward direction within the hierarchical structure. This linking object of type VG and its subordinate linking objects of types VGH, VGH and VGN are used for the grouping of class objects and facilitate a definition of relationships between different classes. Generally speaking, they generate a “grouping hierarchy.”

The linking object of type VÄ and its subordinate linking objects of types VÄH, VÄHA and VÄHJ, VÄN, VÄNA and VÄNJ, which constitute a similarity link, moreover create a relationship having the meaning “is similar in general” in an upward direction within the hierarchical structure and the meaning “is similar in particular” in a downward direction within the hierarchical structure. What takes place here is a hereditary transfer of links to analysis objects and/or processing objects from super-class objects to sub-class objects. Generally speaking, they generate a “similarity hierarchy.”

The processes of hereditary transfer and grouping may be performed separately with the aid of the linking object of type VÄ and its subordinate linking objects of types VÄH, VÄHA and VÄHJ, VÄN, VÄNA and VÄNJ, or by means of the linking object of type VG and its subordinate linking objects of types VGH and VGN.

There is, however, also the possibility of jointly performing the processes of hereditary transfer and grouping, which is carried out with the aid of the linking object of type VÄG and its subordinate linking objects of types VÄGH, VÄGHA, VÄGHJ and VÄGN which have both grouping and heredity properties.

The hierarchies of the class objects that are obtained in this way are either unambiguous, which means that each class object has one super-class object at the most, or they are not unambiguous, which means that each class object may have several super-class objects, and which leads to multiple heredity (similarity hierarchy) or to a multiple semantic pertinence (grouping hierarchy).

The above described linking objects moreover result in a special hierarchical subdivision of the network of class objects. Owing to this subdivision, it becomes possible in particular to formulate more complex semantics for the processing of contents of the data structure to be processed.

In the following it shall be described in what way a classification of semantic structure units is performed.

The structure objects, or generally semantic units, to be classified are initially linked with each respective possible class object via linking objects of type VKA.

Here, two different strategies for carrying out the classification are possible.

The first strategy is that all those class objects qualify for an analysis of respective semantic units to be analyzed, which are located on the bottommost plane of hierarchy of a similarity hierarchy of a network of class objects obtained with the aid of linking objects of type VÄHA, or which are explicitly marked as qualifying. The respective semantic units to be analyzed are linked with these class objects via linking objects of type VKA for the purpose of analysis. Subsequently the analyzed semantic units are allocatingly connected via linking objects of type VKZ with one or several class objects whose feature evaluation yields a pertinence or the highest pertinence to the class represented by a respective class object.

The second strategy is that all those class objects qualify which are arrived at, starting out from the class objects on the topmost plane of hierarchy of a similarity hierarchy in the network of class objects obtained with the aid of linking objects of type VÄ along a hierarchical decision path in the similarity hierarchy. In the case of such a respective class object, this decision path continued in a respective plane of hierarchy of the similarity hierarchy, to which a respective semantic unit to be analyzed exhibits the highest pertinence. The next class objects to be evaluated then are all those class objects linked from the class object having the highest pertinence with the aid of linking objects of type VÄHA in a downward direction in the similarity hierarchy. On this decision path, a respective semantic unit to be analyzed is allocatingly linked, with the aid of a linking object of type VKZ, with the one class object from which there are no further linking objects of type VÄHA in a downward direction in the similarity hierarchy.

Linking objects of type VKA apply the feature objects provided for the analysis object linked with a respective class object (possibly inherited) and the mutual logical links thereof.

In the following, it will be described in what way a local processing is performed.

When a semantic unit is linked with a class object via a linking object of type VKZ, with this class object at the same time being linked with one or several processing objects, then a linking object of type VKP may be generated between the semantic unit and the class object; via this linking object of type VKP, the processing algorithm contained in the processing object(s) is applied to the semantic unit and/or to particular partial networks.

The temporal control process, the time when this takes place, may be predetermined globally for all class objects having a link with a processing object, may be predetermined globally for specific class objects having a link with a processing object, or may be a further constituent of a specific processing object.

With regard to possible modifications within the hierarchical network, reference is made to the above explanations concerning the processing objects.

The following is a description of a cluster analysis of semantic structure units.

With the aid of a customary method, i.e. cluster analysis, semantic structure units in a predetermined feature space may be subsumed into groups of semantic structure units having similar properties for further processing.

Class objects may be generated automatically together with the feature objects pertaining to them, so that they each describe a cluster of similar semantic structure units in a predetermined feature space.

One possibility that is of importance for many applications is the supplementation of information not directly present in the acquired data structure. This is possible thanks to structure objects which do not represent any area of the data structure and may be generated through corresponding analysis and processing prescriptions.

Supplementation of information not directly contained in the data structure may also be effected through transfer of attributes of a class object to semantic units linked thereto.

The following is a description of a processing result.

As a result of a processing, there exist hierarchical networks of semantic structure units where each structure object is linked, via linking objects of type VS, with other structure objects of its environs and was allocated, via linking objects of type VKZ, with either none, one, or several ones of class objects or the class thereby represented, respectively.

Starting out from such class objects, along a hierarchy of connotation by means of linking objects of type VG, further information for each structure object allocated to a class is contained, such as a more general classification via linking objects of type VGH in an upward direction in the hierarchy, or other classes having similar meaning via linking objects of type VGN.

For each class object, all of the semantic units allocated to this class object or to the class thereby represented, respectively, may be retrieved via a link through the intermediary of a linking object of type VKZ.

Along the variegated links of the semantic units, navigation through the semantic network may be carried out, wherein a detail information of interest concerning the analyzed or processed image may be retrieved.

The entire resulting semantic network, and thus the information represented in it with regard to pictorial contents, may be stored and loaded anew.

It furthermore is an essential advantage that by taking out all datapoint objects from a network of structure units after a performed processing of an acquired data structure, a strong reduction of the total information is possible without the necessity of limiting the new information obtained through the generated meaningful interlacing of the network of structure units and of the network of knowledge units. The method is consequently particularly well suited for compressing of data structures or for establishing databases.

In order to elucidate the above explanations, an illustrative example shall be described hereinbelow.

FIG. 11 shows an example for networked environs of a classified structure object 8 and of a corresponding class object 9 in schematic representation. In this figure, respective linking objects between semantic units are symbolically represented as lines interconnecting these semantic units.

The classified structure object 8 is linked with the class object 9 via an allocating linking object of type VKZ. Moreover the classified structure object 8 is linked with other structure objects designated in the figure as “SO” via hierarchical and neighborhood linking objects of type VS. Through its sub-structure objects, the classified structure object 8 is indirectly linked with the datapoint objects 7 designated by “DPO” in the figure, and thus with the acquired data structure that it represents. As is apparent from the figure, the classified structure object 8 that is linked with the class object 9 represents a class that is expressed through the class object 9. Via linking objects of type VÄHA, whereby links to analysis objects and thus feature descriptions may equally be transferred by heredity, the class object 9 is linked with other class objects designated by “KO” in the figure and inherits from these the respective feature descriptions. Thus the class object 9, or class represented thereby, respectively, may be referred to as being similar to other class objects. In addition, the class object 9 is semantically allocated to another class object via a linking object of the type VGH as a subordinate class object. Hereby it is determined that semantically speaking, the class object 9 in general has to be understood as the other class object.

It is an essential point that data structures of different contents, such as, e.g., DNA, m-RNA, t-RNA, r-RNA and proteins, as well as the information items and information flows connected with these, are analyzed both by themselves and in relation with each other at the same time.

For example transition from the DNA to the RNA to proteins is performed not by grouping but by copying and docking objects. The m-RNA is created from the DNA by copying information, the t-RNA is created from the m-RNA by copying information, and the proteins are created by docking amino acids to corresponding positions of the t-RNA by means of enzymes prompted by the t-RNA and docking of the t-RNA to the loaded m-RNA.

Nevertheless the location of copying and of the creation of information and building blocks of the considered data structures is different. The DNA is located in the nucleus, the m-RNA is created by copying from the DNA within the nucleus, the t-RNA is created by copying from the m-RNA in the ribosomes in the cytoplasm, and the proteins are created by docking amino acids to the t-RNA in the cytoplasm and docking the loaded t-RNA to the m-RNA in the ribosomes in the cytoplasm.

The contents, informational content and functions, such as locations of generation and effect of the data structures to be networked, are also different.

As a data source, any data source providing genetic information may serve, such as fluorescence or electrophoresis products.

The respective single data, i.e. the bases A, T, G and C, are entered into a network of semantic structure units which in the present case of this practical example is the DNA network of semantic structure units. By means of a network of semantic knowledge units, semantic structure units and/or their networking are generated on the basis of the acquired data, analyzed, modified, deleted and/or stored in iterative steps. Thanks to interlacing of the DNA, RNA and protein networks of semantic units respectively made up of a network of semantic structure units, this is done not only within a network of semantic units, but in all three of the interlaced DNA, RNA and protein networks. The semantic structure units and/or their networking within the DNA, RNA and protein networks are classified in iterative steps. Based on this classification, specific processing with the aid of the network of semantic knowledge units may be actived, thanks to which various operations may be performed. For example one or several semantic structure units and/or a particular partial network may be changed. These may moreover also be classified by new criteria. Herein the data structures present in the DNA, RNA and protein networks are analyzed on the basis of classifications and networkings both within the DNA, RNA and protein networks and also between these networks, both by themselves and in relationship with each other, at the same time.

In this way, with the aid of the above described processings, in the DNA network of semantic structure units, for example structure units for nucleotides, codogens, genes, operons, chromosomes etc. are formed, changed, networked and/or classified by means of the knowledge base present in the network of semantic knowledge units.

At the same time, by the above described processings in the RNA network of semantic structure units, for example structure units for nucleotides, codons, genes, operons, chromosomes etc. are formed, modified, networked and/or classified by means of the knowledge base present in the network of semantic knowledge units.

Finally by the above described processings in the protein network of semantic structure units, for example structure units for amino acids, proteins, groups of proteins etc. are formed, modified, networked and/or classified by means of the knowledge base present in the network of semantic knowledge units.

Networking within the networks and between the networks has a hierarchical structure, with neighborhood relationships also existing, however.

As a result of networking the networks of semantic structure units and the networks of semantic knowledge units, processing of the information present in the different data structures is made possible which is selective and adapted to the respective set of problems. The data structures here act both on the networks of semantic structure units and on the network of semantic knowledge units. Vice versa, these networks also act on the data structures. The different data structures moreover act on each other and on the respective other networks. In this way, artefacts contained in the data structures may be remedied, knowledge about the genetic code may be corrected, e.g. by simulation, and supplemented, newly acquired, or even newly generated.

To be more precise, the networks of semantic structure units for DNA-, RNA- and protein data structures have a hierarchical structure such that based on different hierarchy levels an organism or several organisms may be represented. Data may be input at least into a partial area of one or several hierarchy levels of one or several of the networks of semantic structure units. With the aid of the classification, a functional analysis of semantic structure units is performed in dependence on input data, such that a respective function of semantic structure units and of their networkings is determined and/or changed by all networks and between all networks of semantic structure units, and as a result of the functional analysis, the knowledge base present in the network of semantic knowledge units possibly will be changed.

The change of the knowledge base may, for example, comprise a deleting, supplementing and/or changing of semantic knowledge units and of their networkings within the network of semantic knowledge units and between the semantic networks of knowledge units and respective networks of semantic structure units.

The above described function analysis serves diagnostic purposes so as to determine causes of changes inside an organism or several organisms.

For example there is the possibility of determining possible genetic causes for a cancerous affection for a particular organism. If the same kind of cancerous affection also occurs in other organisms, possible causes may also be determineed for these organisms. By comparison of the respectively ascertained causes, there is then a possibility of determining what possible causes were ascertained in all of the organisms. These causes ascertained in all organisms may then be classified as causes that are responsible for the cancerous affection with a high probability or certainty. Based on the classification, the knowledge base present in the network of semantic knowledge units may then be changed, i.e., changed as to contents, expanded, or restricted.

The following is an exmplary enumeration of semantic units that may be defined in the DNA, RNA and protein networks.

For example, in all of the three above mentioned networks the following semantic units may generally be defined: objects known from biology, such as nucleotides, codogens or codons, genes, structure genes, operator genes, operons, regulator genes, DNA, m-RNA. t-RNA, amino acids, proteins, enzymes, peptides etc., and objects whose definition proves necessary as a result of the set of problems, such as a portion of an operon, so as to be able to deal with a respective set of problems.

In the DNA network, for example, the following semantic units may generally be defined: nucleotides, codogens, genes (the DNA portions comprised of approximately 10³ nucleotide pairs, with their structural pattern containing the information for a polypeptide chain), operator genes (the portion at the beginning of a series of structure genes which determines functionally co-operative proteins. The operator gene starts the formation of the m-RNA at this series of genes where not prevented by a reated repressor.), operons (regulatory units from a number of structure genes, an operator gene located at the beginning, and a regulator gene mostly located in another position. The series of structure genes carries the information for biochemically cooperating enzymes, for example for the steps of synthesis of an amino acid. Formation of all enzymes determined by an operon is disabled by the repressor in that the latter suppresses the formation of a related m-RNA, regulator genes, structure genes, DNA and objects which are defined by an automatic classification during an analysis and appear meaningful for dealing with a respective set problem, wherein a result of the classification may be whether or not these objects are meaningful in the sense of the set problem.

In the RNA network, for example, for the m-RNA and the t-RNA respective similar semantic units as for the DNA may generally be defined.

In the protein network, for example, the following semantic units may generally be defined: amino acids (these are the twenty most important, naturally occurring ones as known from biology, namely, glycine, alanine, valine, leucine, isoleucine, serine, threonine, asparagic acid, aspartamic acid, glutamic acid, glutamine, lysine, arginine, histidine, phenylanaline, tyrosine, tryphtophane, proline, cysteine, methionine.), groups of amino acids (peptides, such as dipeptides, tripeptides, polypeptides), enzymes, proteins (as groups of more than one hundred amino acids) and objects which are defined during an analysis or by automatic classification and appear meaningful for working on a particular set problem, wherein it may be an outcome of the classification whether or not these objects are meaningful in the sense of the set problem.

The following definitions of relationships may generally exist in all three networks:

Definitions concerning a neighborhood, such as, e.g., what neighbors and what distance from the neighbors exist. A distance between semantic units in the DNA network may, for example, be a length distance between identical codogens or similar codogens. In general, a distance may be a length distance between semantic units. Moreover a distance may, for example, be a similarity distance or similarity difference, such as a difference between contents, i.e. sub-objects, between semantic units. Finally, a distance may be defined as a position of a semantic unit within the network of semantic units.

Definitions concerning a similarity, such as, e.g., codon_{—}1 is similar to codon_{—}2.

Definitions as to what sub-objects, for example what sub-objects a particular codon has, what codons are within an operon or what codons are located within a semantic unit defined by an analysis or by a classification with respect to a set problem.

Definitions as to what super-objects, for example what nucleotide to what codon, what codon to what operon, or what semantic unit has what other ones as a super-object within a respective network.

Definitions as to what relationships with the super-object, for example which welche position with respect to the entire super-object does the sub-object have, whereby, for example, a codon START or STOP is defined, or what position does a codon have within an operon, such as third from the end or third from the beginning.

Definitions as to what relationships with the sub-object such as codon_{—}1, for example, does a particular characteristic property of operon_{—}1 define, or a missing nucleotide within a structure gene scrambles the meaning of the entire structure gene.

Relationships defined either by a user with regard to a set problem or automatically by a classification, or which prove necessary, which do not correspond to previously existing biological knowledge.

Kinds of relationships moreover are relationships between the three networks which serve for information flows between the networks with regard to a set problem, e.g., copy of codon_{—}1 in the m-RNA is a copy of codogen_A in DNA, association with, e.g., amino acid_A is associated with codon_{—}1 in the m-RNA, responsible for, e.g., structure gene_A is responsible for tripeptide B in the protein network, derived from, e.g., error on gene_{—}1 may or might be derived fromr the erroneous information supplied from the m-RNA via codon_{—}2, wherein the error on codon_{—}2 may or might in turn be derived from the erroneous information supplied by codon of the t-RNA upon prompting an enzyme_G instead of an enzyme D during docking of amino acid_C during formation of the protein_{—}1, formation via, e.g., unknown kind protein_A might be formed via lack of codon_{—}1 in gene_{—}1 of the DNA, and carrier of information, e.g., via the formation of polypeptide G.

Features and attributes are moreover defined for a classification by meansa of string-matching, e.g., strings texture content, length, variance, texture, position. Possibly attributes of effect may also be defined, e.g., codon_{—}1 corresponds to START or STOP.

All of the above described relationships in turn are semantic units.

As was mentioned above, apart from semantic units having a meaning with regard to genetic, biological, bio-medical and biochemical data structures such as DNA, RNA and proteins, semantic units may furthermore be generated and employed in a further processing, which do not have any known meaening with regard to the known interconnections in biology, biochemistry, bio-medicine and genetics. The like semantic units are classified as fictitious semantic units and may be used in a respective set problem where this proves to be meaningful. For example, a semantic unit initially classified to be ficticious may during further processing be classified again to finally be a semantic unit having a meaning with regard to the biological, biochemical, bio-medical and genetic data structures.

The fictitious semantic units may both be located in one hierarchy level wherein semantic units carrying a meaning with regard to the structures are present, and may also define intermediate levels in the hierarchy wherein no semantic units carrying a meaning with regard to the data structures are present.

By referring to FIGS. 12 to 17 hereinbelow an example of classified networks of semantic structure units of DNA, RNA and protein data structures will be given by concurrently representing a class hierarchy.

FIG. 12 shows an example of a classified hierarchical network of structure units of a DNA data structure in accordance with the practical example of the present invention.

As may be seen in FIG. 12, the classified network of semantic structure units contains four hierarchy levels, i.e., the level of nucleotides, the level of codogens, the level of operons, and the level of genes. On different levels there equally exist semantic structure units not having any meaning with regarrd to the DNA data structure and consequently being classified as fictitious semantic units. The relationships realized by means of linking objects are represented in FIG. 12 by lines between respective semantic structure units.

FIG. 13 shows a representation of class objects in one grouping hierarchy in accordance with the DNA data structure in FIG. 12, with representation being effected in FIG. 13 from the left to the right from the topmost to the bottommost hierarchy level in FIG. 12.

FIG. 14 shows an example of a classified hierarchical network of structure units of an RNA data structure in accordance with the practical example of the present invention.

As may be seen in FIG. 14, the classified network of semantic structure units contains four hierarchy levels, i.e., the level of nucleotides, the level of codons, the level of operons, and the level of genes. On different levels there also exist semantic structure units not having any meaning with regard to the RNA data structure and consequently being classified as fictitious semantic units. The relationships realized through linking objects realisierten are represented in FIG. 12 by linees between respective semantic structure units.

FIG. 15 shows a representation of class objects in one grouping hierarchy in accordance with the RNA data structure in FIG. 14, with representation in FIG. 15 being effected from the left to the right from the topmost to the bottommost hierarchy level in FIG. 14.

FIG. 16 shows an example of a classified hierarchical network of structure units of a protein data structure in accordance with the practical example of the present invention.

As may be seen in FIG. 16, the classified network of semantic structure units contains three hierarchy levels, i.e., the level of amino acids, the level of peptides, and the level of proteins. On different levels there equally exist semantic structure units which do not have any meaning with regard to the protein data structure and consequently are classified as fictitious semantic units. The relationships realized with the aid of linking objects are represented in FIG. 16 by lines between respective semantic structure units.

FIG. 17 shows a representation of class objects in one grouping hierarchy in accordance with the protein data structure in FIG. 16, wherein representation in FIG. 16 is effected from the left to the right from the topmost to the bottommost hierarchy level in FIG. 16.

Although this is not represented in FIGS. 12 to 17, the three networks of semantic structure units of the DNA, RNA and protein data structures are furthermore interlaced with each other by means of a coupling network made up of linking objects. This is, for example, carried out in such a way that, e.g., superordinate semantic structure units have subordinate structure units from different networks of semantic structure units. Moreover neighborhood and similarity relationships between semantic structure units may exist.

For example, codon_{—}1 in the DNA (FIG. 12) may have a relationship of “codon_{—}1 in DNA is copy of codogen_{—}1” with codogen_{—}1 in the RNA (FIG. 13). Such relationships may exist between all networks of semantic structure units and ensure flow of information in any directions whatsoever.

With regard to further features and advantages of the present invention, reference is expressly made to the disclosure of the drawings.

1. A method for processing data structures of different structures and contents by means of networked semantic units, which method includes the steps of:
[a] acquisition of data wherefrom the different data structures are derivable, wherein respective data structures are represented in respective networked networks of semantic structure units; and
[b] generating, analyzing, modifying, deleting and/or storing the semantic structure units and/or networking them based on the acquired data by using a knowledge base comprised of a network of semantic knowledge units, wherein
in iterative steps semantic structure units and/or the networking thereof are classified and a specific processing may be activated thanks to this classification, which specific processing modifies a respective semantic structure unit and/or a particular partial network.

2. The method in accordance with claim 1, characterized in that each of the semantic networks of structure units includes a hierarchy with hierarchy levels of superordinate, subordinate and neighboring semantic structure units.

3. The method in accordance with claim 2, characterized in that superordinate semantic structure units comprise subordinate structure units from different networks of semantic structure units.

4. The method in accordance with claim 2 or 3, characterized in that data is acquired in one or several arbitrary hierarchy levels.

5. The method in accordance with any one of the preceding claims, characterized in that the networks of semantic structure units overlap each other.

6. The method in accordance with any one of the preceding claims, characterized in that in step [b] a networking between networks of semantic structure units is changed.

7. The method in accordance with any one of the preceding claims, characterized in that in step [b] a networking between the network of semantic knowledge units and the networks of semantic structure units is changed.

8. The method in accordance with any one of the preceding claims, characterized in that the several different data structures are interrelated biological, biochemical, biomedical and/or genetic data structures.

9. The method in accordance with claim 8, characterized in that the several different data structures are DNA data structures, RNA data structures and/or protein data structures that are each represented in a network of semantic structure units.

10. The method in accordance with claim 9, characterized in that networks of semantic structure units for DNA-, RNA- and protein data structures have a hierarchical structure such that based on different hierarchy levels an organism or several organisms may be represented, wherein data may be input at least into a partial area of one or several hierarchy levels of one or several of the networks of semantic structure units, and with the aid of the classification, a functional analysis of semantic structure units is performed in dependence on input data, such that a respective function of semantic structure units and of their networkings is determined and/or changed by all networks and between all networks of semantic structure units, and as a result of the functional analysis, the knowledge base present in the network of semantic knowledge units possibly will be changed.

11. The method in accordance with claim 10, characterized in that function analysis serves diagnostic purposes so as to determine causes of changes inside an organism or several organisms.

12. The method in accordance with any one of claims 9 to 11, characterized in that the classification is carried out by comparing, allocating, feeding back and deriving the several different data structures in such a manner that
respective networks of semantic structure units of the several different data structures are classified by means of the network of semantic knowledge units themselves and in dependence on this classification relationships and networkings within the respective networks of semantic structure units of the several different data structures are generated, and
the respective networks of semantic structure units of the several different data structures are classified with respect to each other by means of the network of semantic knowledge units, and relationships and networkings between the respective networks of semantic structure units of the several different data structures are generated in dependence on this classification.

13. The method in accordance with claim 12, characterized in that networking between the respective ones of the networks of semantic structure units of the several different data structures takes place with the aid of a classification of information flows from a network of semantic structure units of the DNA data structures to a network of semantic structure units of the RNA data structures to a network of semantic structure units of the protein data structures and similar regressive processes.

14. The method in accordance with any one of claims 8 to 13, characterized in that semantic structure units are structure objects, linking objects linking semantic structure units, or networks/partial networks of semantic structure units.

15. The method in accordance with claim 14, characterized in that the semantic structure objects are present in a super-structure object hierarchy, sub-structure object hierarchy and neighbor-structure object hierarchy in the networks of semantic structure units, with respective linking objects defining respective relationships between respective structure objects.

16. The method in accordance with claim 15, characterized in that the relationships include neighborhood relationships, similarity relationships, relationships with super-structure objects, and relationships with sub-structure objects.

17. The method in accordance with any one of claims 9 to 16, characterized in that DNA sequences in a network of semantic structure units of DNA data structures are classified in that nucleotide structure units are grouped in sequences of three into newly formed codogen structure units and relationships between semantic structure units are formed, wherein the relationships include neighborhood relationships, similarity relationships, relationships with super-structure objects and relationships with sub-structure objects.

18. The method in accordance with any one of claims 9 to 17, characterized in that RNA sequences are classified in a network of semantic structure units of RNA data structures in that nucleotide structure units are grouped in sequences of three into newly formed codon structure units and relationships between semantic structure units are formed, wherein the relationships include neighborhood relationships, similarity relationships, relationships with super-structure objects and relationships with sub-structure objects.

19. The method in accordance with any one of claims 9 to 18, characterized in that relationships between DNA sequences in a network of semantic structure units of DNA data structures and RNA sequences in a network of semantic structure units of RNA data structures are formed by semantic structure units describing an allocation of semantic DNA structure units to semantic RNA structure units.

20. The method in accordance with any one of claims 9 to 19, characterized in that a classification of semantic structure units describing amino acids is carried out in a network of semantic structure units of protein data structures in such a manner that the semantic structure units describing amino acids are grouped, semantic structure units for the formed amino acid groups are formed, and relationships between the semantic structure units are formed, wherein the relationships include neighborhood relationships between the semantic structure units describing amino acids and similarity relationships between the semantic structure units describing amino acids, and wherein semantic structure units are generated and defined which describe relationships with semantic enzyme structure units, and semantic structure units are generated which describe relationships with super-structure objects.

21. The method in accordance with claim 20, characterized in that the super-structure objects are semantic protein structure units.

22. The method in accordance with any one of claims 9 to 21, characterized in that local processes are classified as semantic units describing processes of a transformation of biological information.

23. The method in accordance with claim 22, characterized in that the local processes are described by:
copying a DNA structure unit to an RNA structure unit;
fusing semantic structure units for nucleotide triplets into semantic structure units for an operon;
separating semantic units for nucleotide triplet groups into single semantic structure units for a nucleotide triplet;
regrouping semantic structure units for nucleotide triplets of a semantic structure unit for an operon into another semantic structure unit for an operon;
deleting semantic structure units for nucleotide triplets within a semantic structure unit for an operon; and
allocating semantic structure units for nucleotide triplets to semantic structure units for an operon.

24. The method in accordance with any one of claims 9 to 23, characterized in that local processes are classified as semantic units describing processes of a transformation of biochemical and genetic information.

25. The method in accordance with claim 24, characterized in that the local processes are described by:
allocating semantic structure units for amino acids to a semantic structure unit for an m-RNA;
fusing semantic structure units for amino acids into higher semantic structure units within a protein and generating its allocation to semantic structure units for metabolic processes;
separating semantic structure units for amino acid groups into single semantic structure units for an amino acid;
regrouping semantic structure units for amino acids or amino acid groups;
deleting semantic structure units for amino acids within a partial network describing a semantic structure unit for a protein;
allocating semantic structure units for amino acids to the semantic structure units for proteins; and
forming semantic linking objects between the semantic structure units for amino acids, the semantic structure units for amino acid groups, and the semantic structure units for proteins.

26. The method in accordance with any one of claims 9 to 25, characterized in that semantic linking objects are formed which describe an allocation of a semantic structure unit for a t-RNA to different amino acids in protein formation.

27. The method in accordance with any one of claims 9 to 26, characterized in that semantic structure units are formed which describe local processes of copying, of searching, of prompting, of selecting, and of docking as semantic processing objects.

28. The method in accordance with any one of claims 9 to 27, characterized in that the following processes may be performed within a data structure:
fusing two or more codon structure units into a new super-structure unit, wherein at the same time the nucleotide structure units pertaining thereto are divided and new neighborhood relationships between these are generated;
fusing two or more codon or operon structure units into a larger structure unit by generating a new hierarchy level, wherein the corresponding codon or operon structure units form the sub-structure units of this new, larger structure unit;
incorporating two or more new structure units within a network of semantic structure units in such a way that upon generation of a new incorporation, a structure unit becomes a sub-structure unit of another structure unit;
separating a structure unit from its super-structure unit by generating a new kind of a neighborhood relationship with the former super-structure unit;
newly allocating a structure unit to a new sub-structure unit; and
regrouping a structure unit from one group of structure units to another group of structure units.

29. The method in accordance with any one of the preceding claims, characterized in that a classification algorithm is started by string-matching, wherein knowledge units are characterized by particular strings and the classification algorithm recognizes fragments in the several different data structures having identical or similar strings, and generates classification connections with corresponding knowledge units of semantic structure units which correspond to these fragments, whereby in turn new algorithms may be called up.

30. The method in accordance with claim 29, characterized in that classification is carried out with the aid of attributes.

31. The method in accordance with claim 30, characterized in that the attributes include the strings code, content, length, variance, texture and position.

32. The method in accordance with any one of the preceding claims, characterized in that classification is carried out with the aid of neighborhood relationships.

33. The method in accordance with claim 32, characterized in that the neighborhood relationships include what neighbors, what relationship with the neighbors, what super-units, what sub-units and what relationships with the super-units and/or sub-units exist.

34. The method in accordance with any one of the preceding claims, characterized in that semantic structure units are generated which are merely allocated a fictitious meaning in a classification by means of the network of semantic knowledge units.

35. The method in accordance with claim 34, characterized in that the semantic structure units having merely been allocated a fictitious meaning are taken into consideration in another classification.
